(19) United States
(12) Patent Application Publication
(10) Pub. No.: US 2023/0384189 A1
(43) Pub. Date: Nov. 30, 2023

(54) MICROCAPILLARY DEVICE, DETECTION DEVICE, AND METHODS RELATED THERETO

(71) Applicants: Mahla Poudineh, Etobicoke (CA); Fatemah Keyvani, Waterloo (CA); Nandini Debnath, Edmonton (CA)

(72) Inventors: Mahla Poudineh, Etobicoke (CA); Fatemah Keyvani, Waterloo (CA); Nandini Debnath, Edmonton (CA)

(21) Appl. No.: 18/309,141

(22) Filed: Apr. 28, 2023

Related U.S. Application Data
(60) Provisional application No. 63/337,051, filed on Apr. 30, 2022.

(51) Int. Cl. G01N 1/34; C12Q 1/6825; B01L 3/00; G01N 27/327

(52) U.S. Cl. CPC G01N 1/34; C12Q 1/6825; B01L 3/502761; G01N 27/3276; B01L 2300/0645; B01L 2300/0681; B01L 2300/0864; B01L 2400/0406; B01L 2200/0652

(57) ABSTRACT

A microfluidic passive sample separation device for passively separating a liquid sample, and optionally detecting an analyte in the separated liquid sample. The device includes a filter for filtering the liquid sample and for producing a filtrate; a plurality of capillaries configured to withdraw the filtrate from the filter by capillary force, a capillary micropump for receiving the filtrate and pumping the filtrate to the outlet.

### CROSS REFERENCE TO RELATED APPLICATION

This application claims priority to U.S. Provisional Patent Application No. U.S. 63/337,051 filed Apr. 30, 2022, the entire contents of which are hereby incorporated by reference.

### SEQUENCE LISTING

The instant application contains a Sequence Listing which has been submitted electronically in XML format and is hereby incorporated by reference in its entirety. Said XML copy, created on Apr. 28, 2023, is named BLH-00601_Sequence-_Listing.xml and is 5 kb in size.

### FIELD

The present disclosure generally relates to a microcapillary device and method for passively separating and transporting components of a liquid sample; a sensor and method for detecting an analyte of interest in a liquid sample; and an integrated device and method for separating components of a liquid sample and detecting an analyte of interest. In particular, the present disclosure describes devices and methods that are suitable for use at point-of-care (POC).

### BACKGROUND

Although various microfluidic sample separation devices and assays are known, most require some combination of sample preparation and/or processing, external forces to pump fluids, and special equipment. Such steps and equipment add to cost and also limit use at point-of-care (POC).

Although various sensors, such as electrochemical detection devices and assays are known, improvements are needed, in particular for detecting complex analytes, such as biological molecules and/or for detecting analytes in small-volume liquid samples without requiring analyte processing steps and/or special equipment.

An integrated microfluidic passive sample separation and sensor, such as an electrochemical detection device that requires no sample processing and no special equipment, such that it can be used at point-of care (POC) (used interchangeably herein with point-of-use), would advance the state of the art in the field of diagnostics. In particular, a device that could effectively separate plasma from whole blood samples and/or permit detection of complex analytes, such as biological molecules, at POC.

### SUMMARY OF VARIOUS EMBODIMENTS

Outlined below are various embodiments of the invention. It should be understood that the embodiments so described are non-limiting and that individual features and components may be combined in any combination or order that provides a useful device or method according to the invention. Once armed with the teachings herein, persons skilled in the art will be able to select combinations of features according to their particular needs while still falling within the scope of the present invention.

In one aspect, there is provided a microfluidic passive sample separation device comprising: a body defining an inlet and an outlet, the body housing a filter for filtering a liquid sample deposited thereon and for producing a filtrate; a plurality of capillaries configured to withdraw the filtrate from the filter by capillary force, each capillary having a first end fluidly connected to the filter for receiving the filtrate; and a second end fluidly connected to a capillary micropump; the capillary micropump being configured to receive the filtrate and to pump the filtrate through the outlet.

In some embodiments, the plurality of capillaries are substantially parallel. In some embodiments, each capillary has a width and is spaced about three times the width from an adjacent capillary. In some embodiments, each capillary has a width of about 12 μm and is spaced about 40 μm from an adjacent capillary.

In some embodiments, the capillary micropump has at least a first end and a second end opposite the first end, and wherein the width of the micropump increases and then decreases between the first end and the second end. In some embodiments, the capillary micropump is an elliptical, half-hexagonal, or hexagonal capillary micropump. In some embodiments, the capillary micropump is a hexagonal capillary micropump. In some embodiments, the capillary micropump comprises a staggered array of substantially circular-, square-, rectangular-, or oval-shaped microstructures. In some embodiments, the capillary micropump comprises a staggered array of substantially oval-shaped microstructures.

In some embodiments, the filter is a filter membrane. In some embodiments, the filter membrane is sealed to the inlet. In some embodiments, filter membrane has a thickness of about 9 μm. In some embodiments, the filter membrane has a pore size of about 0.6 μm.

In some embodiments, the microfluidic passive filtration device is inverted.

In some embodiments, the microfluidic passive filtration device further comprises a sensor for detecting an analyte, the sensor being fluidly connected to the microfluidic passive filtration device. In some embodiments, the sensor is an electrochemical biosensor as described herein.

In another aspect, there is provided an electrochemical biosensor.

In some embodiments, the electrochemical biosensor comprises a working electrode having a surface comprising a probe configured to bind, couple or hybridize with the analyte. In some embodiments, the working electrode is a carbon-based electrode, gold-based electrode or a graphene-based electrode. In some embodiments, the working electrode is a carbon electrode. In some embodiments, the working electrode is a screen-printed carbon electrode (SPCE). In some embodiments, a probe is immobilized on the working electrode by drop-casting graphene oxide (GO) on the surface of the working electrode for forming a covalent bond between the probe and the GO. In some embodiments of the devices and methods of the present invention, the electrochemical biosensor comprises a graphene-oxide screen-printed carbon electrode (GO-SPCE) probe chip.

In another aspect, there is provided an integrated separation and detection device for detecting an analyte in a liquid sample, the device comprising: a microfluidic passive sample separation device comprising: a body defining an inlet and an outlet, the body housing; a filter for filtering a liquid sample deposited thereon and for producing a filtrate; a plurality of capillaries configured to withdraw the filtrate from the filter by capillary force, each capillary having a first end fluidly connected to the filter for receiving the filtrate; and a second end fluidly connected to a capillary micropump for dispensing the filtrate to the capillary micropump; the capillary micropump being configured to receive the filtrate and to pump the filtrate through the outlet; and an electrochemical biosensor, the electrochemical biosensor fluidly coupled to the microfluidic passive sample separation device for detecting the analyte in the filtrate.

In another aspect, there is provided a method of separating a liquid sample, the method comprising; passing the liquid through a filter to form a filtrate; passing the filtrate through a plurality of capillaries for drawing the filtrate through the filter by capillary force; pumping the filtrate by capillary micropump for pumping the filtrate through the plurality of capillaries. In some embodiments, method comprises inverting the microfluidic passive filtration device.

In some embodiments, the method further comprises transporting a filtrate to an electrochemical biosensor and contacting the filtrate with a probe for detecting an analyte for a sufficient time and under suitable conditions to detect the analyte. In some embodiments, the electrochemical biosensor is described herein.

In another aspect, there is provided a method of detecting an analyte in a liquid sample comprising: passing the liquid sample through a filter to form a filtrate; passing the filtrate through a plurality of capillaries for drawing the filtrate through the filter by capillary force; pumping the filtrate by capillary micropump to an electrochemical biosensor, the electrochemical biosensor comprising a probe configured to detect the analyte; contacting the filtrate with the probe for a sufficient time and under suitable conditions to detect the analyte; and detecting the presence of the analyte by electrochemical means.

In some embodiments, the method further comprises incubating the GO-SPCE probe chip at about 37° C. following the screening. In some embodiments, the GO-SPCE is incubated for about 10 to about 60 minutes. In some embodiments, the method further comprises washing the incubated GO-SPCE probe chip with a buffer. In some embodiments, the method further comprises sedimenting the liquid sample prior to filtering the liquid sample. In some embodiments, the liquid sample is inverted prior to filtering.

In another aspect, there is provided a method of detecting an analyte in a whole blood sample comprising: passing the whole blood sample through a membrane filter to form a filtrate comprising plasma; passing the filtrate through a plurality of substantially parallel capillaries to isolate the plasma from the filtrate by capillary force; pumping the plasma by capillary micropump to an electrochemical biosensor, the electrochemical biosensor comprising a probe specific for the analyte; contacting the filtrate with the probe for a sufficient time and under suitable conditions for detecting the analyte using the electrochemical biosensor; and detecting the presence of the analyte by electrochemical means.

In some embodiments, the electrochemical biosensor comprises a graphene-oxide screen-printed carbon electrode (GO-SPCE) probe chip.

In some embodiments, the method further comprises sedimenting the whole blood sample prior to filtering the whole blood sample. In some embodiments, the method further comprises incubating the GO-SPCE probe chip at about 37° C. following the screening. In some embodiments, the GO-SPCE is incubated for about 10 to about 60 minutes. In some embodiments, the method further comprises washing the incubated GO-SPCE probe chip with a buffer.

In accordance with the various aspects or embodiments, the analyte may be any analyte of interest capable of being detected in accordance with the present invention. In some embodiments, the analyte is a biological molecule. In some embodiments, the analyte is a nucleic acid, protein, antibody, antigen, or fragment thereof. In some embodiments, the antigen is a metabolite. In some embodiments, the analyte is a nucleic acid molecule, such as a DNA or RNA molecule, or a fragment thereof. In some embodiments, the analyte is DNA. In some embodiments, the analyte is circulating DNA (cDNA). In some embodiments, the analyte is an analyte for detecting disease, such as cancer, such as cervical cancer. In some embodiments, analyte is hr-HPV16 cDNA.

In accordance with the various aspects or embodiments, the liquid sample may be any suitable sample capable of being separated in accordance with the present invention. In some embodiments, the sample comprises an aqueous fluid such as water or blood. In some embodiments, the sample comprises blood. In some embodiments, the sample comprises whole blood. In some embodiments, the sample is blood. In some embodiments, the sample is whole blood. When the sample is blood, the filtrate comprises plasma. The sample volume may be any suitable volume. In some embodiments, the liquid sample is about 80 μL to about 200 μL in volume. In some embodiments, the liquid sample is about 80 μL in volume.

In accordance with the various aspects or embodiments, the probe (or probes) may be any suitable probe capable of detecting an analyte of interest in accordance with the devices and methods of the present invention. In some embodiments, the probe is for detecting an analyte as described herein. In some embodiments, the probe is for detecting a nucleic acid analyte. In some embodiments, the probe is cssDNA. In some embodiments, the probe is for detecting hr-HPV16 cDNA.

In some embodiments, the devices and methods described herein are suitable for use at point-of-care (POC). POC may be used interchangeably with point-of-use depending on the sample and purpose of the particular embodiment.

Other features and advantages of the present application will become apparent from the following detailed description, taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described.

FIG. 1 Non-limiting overview and working mechanisms of an exemplary detection device—an integrated microfluidic electrochemical assay (IMEA) for cancer detection (IMEAC). Side view (a) and top view (b) of an IMEAC device that integrates two main modules: PPS for plasma isolation and an electrochemical biosensor. (c) Working principle of the electrochemical sensor for hr-HPV16 cDNA detection. (d) a sample graph extracted from IMEAC showing the presence of hr-HPV cDNA in extracted plasma sample.

FIG. 2 Exemplary microfluidic passive sample separation (PSS) device for plasma separation (PPS) design overview and characterization. (a) PPS device consists of three main modules: membrane filter, parallel capillaries, and capillary micropump. A commercial plasma filter membrane with 0.6 μm average pore size is sandwiched between two PDMS layers at the inlet of the PPS device. The parallel capillaries comprise of micro-channels which are 12 μm width and 40 μm apart from each other. Capillary micropumps consist of staggered oval microarray with 150 μm length and 100 μm width and they have uniform centre to centre distance of 300 μm in horizontal direction and 200 μm in the vertical direction. Scale bar, 100 μm. (b) Comparison of the collected plasma volume for different micropump designs (square, rectangular, circular, oval) for the same inlet blood volume. (c) Comparison of collected plasma volume for no sedimentation (noninverted) vs sedimentation (inverted) for different inlet blood volume showing sedimentation improved plasma collection. (d) Comparison of collected plasma volume for no sedimentation (noninverted) (i) vs sedimentation (inverted) (ii) inside micropipette tips. (e) Inlet whole blood vs combined collected plasma from 8 PPS devices. Each experiment was repeated three times and error bars show SD.

FIG. 3 depicts optimization of the shape of structures incorporated inside the microfluidic PSS device micropump design based on collected plasma volume.

FIG. 4 depicts flow cytometry measurements for calculating the purity of plasma collected from the PPS device.

FIG. 5 Design and characterization of the electrochemical biosensor for hr-HPV16 cDNA detection. (a) The FTIR measurement of GO samples before and after probe immobilization. (b) The CV measurement of GO functionalized with probe in 10 mM Fe(CN)₆^{3-/2-} solution showed a lower oxidation peak compared with GO prior to the probe immobilization. (c) Sample DPV measurement before (dotted line) and after (solid line) specific target hybridization at concentration of 10 μM. The inset shows the DPV measurement of non-specific target (rDNA) at 5 μM concentration. Iss and Ids represent the DPV peak current before and after target hybridization, respectively. (d) Change in DPV current at different target DNA concentrations can be fitted with a linear regression line (R²=0.97). (e) The performance of the electrochemical biosensor was studied at different conditions using 5 μM high risk human papilloma virus 16 (hr-HPV16) cDNA. (f) The performance of the electrochemical biosensor was studied 3 or 7-day post-fabrication using 5 μM hr-HPV16 cDNA. The obtained biosensor responses were normalized to the response of biosensors fabricated and tested in same day, ns=non-significant. At least 3 replicates were analysed per experiment and error bars show SEM.

FIG. 6 depicts DPV scans before and after incubating the GO-SPCE sensors with (a) 0 μM, (b) 1 μM, (c) 2 μM, and (d) 5 μM of hr-HPV16 cDNA.

FIG. 7. Fabrication and validation of exemplary IMEAC integrated detection device comprising exemplary microfluidic PSS device PPS. (a) IMEAC was fabricated via connecting the outlet of the PPS device to the inlet of a chamber that covers the electrodes of the electrochemical biosensor using tubing. (b) A demonstration of assay with food dye. (c) The efficiency of the PPS device to recover DNA molecules was tested by loading a whole blood sample spiked with different concentrations of rDNA labeled with Cy3 dye into the device Background is 0 μM concentration. The fluorescence intensity of the collected plasma was measured and compared with the control plasma spiked with rDNA. (d) DPV measurement before and after incubating with 10 μM hr-HPV16 cDNA spiked plasma extracted from PPS. (e) Bar graph extracted from IMEAC or control plasma demonstrating the change in DPV current for 5 μM and 10 μM of target DNA spiked into whole blood. Each experiment was repeated at least three times and error bars show SEM.

FIG. 8. Overview and working mechanisms of another exemplary Microfluidic Passive Sample Separation Device (PS-V2) and integration with a biosensor. (a) Schematic of the PS-V2 device (side view). The device consists of three PDMS layers; The bottom layer (PDMS Layer 1) contains a straight capillary microchannel (W=600 μm, H=25 μm). The middle layer (PDMS Layer 2>5 mm thickness) consists of a 6.3 mm inlet well (W1) for loading blood and a 1.2 mm outlet well (W2) which connects to the top fluidic microchannels (PDMS Layer 3). The enlarged image shows the schematic of the self-built-in filter in the 1.2 mm well (W2) due to sedimentation and wettability gradient. PDMS Layer 3 houses the capillary design for passive separation, such as for automatic plasma collection and outlet reservoirs. A membrane is bonded between layer 2 and 3. (b) Top view of layer 3 showing the capillary design details comprising of parallel capillaries, capillary micropump and outlet reservoirs. The scale bar is 1.5 mm. (c) PS-V2 device assembly after bonding with membrane. The enlarged image shows the micro capillaries. The scale bar is 150 μm. (d) Demonstration of plasma isolation by manual syringe withdrawing in the integrated system that connects PS-V2 for plasma isolation with a biosensor.

FIG. 9. Collection of plasma using PS-V2 device from whole blood. (a) Top view of the PS-V2 device after 1 min of loading whole blood at inlet. (b) Side view of the PS-V2 device after 10 min of blood loading showing the formation of blood-plasma interface due to sedimentation and capillary action, otherwise referred to herein as wettability gradient. (c) Collected Plasma in a pipette at the outlet of PS-V2 device after 15 min of blood loading.

FIG. 10. Flow cytometry analysis of blood samples vs collected plasma before and after filtration using PS-V2 device. (a) Blood sample and (b), (c), (d) filtered plasma samples with same gating. Blood cells (center black dot) and platelets (black scatter) are gated on forward scatter (FSC-H) and side scatter (SSC-H).

Further aspects and features of the embodiments described herein will appear from the following description taken together with the accompanying drawings.

### DETAILED DESCRIPTION OF THE EMBODIMENTS

The present disclosure relates to a microcapillary device and method for passively separating and transporting components of a liquid sample; an electrochemical biosensor and method for detecting an analyte of interest in a liquid sample; and an integrated device and method for separating components of a liquid sample and detecting an analyte of interest. In particular, the present disclosure describes devices and methods that are suitable for use at point-of-care (POC).

### Passive Sample Separation (PSS) Device

Described herein is a microfluidic passive sample separation (PSS) device. The microfluidic PSS device may be used to separate a sample to form a filtrate. The filtrate may comprise an analyte to be detected. The microfluidic PSS device may separate the sample passively, such that minimal to no forces external to the device need to be applied to facilitate the separation. When separated using the microfluidic PSS device, the sample may be introduced into the device without any pre-processing steps, such as pre-filtering or pre-concentrating steps.

In an embodiment, the device comprises a body defining an inlet and an outlet, the body housing a filter for filtering a liquid sample in contact therewith and for producing a filtrate; a plurality of capillaries configured to withdraw the filtrate from the filter by capillary force, each capillary having a first end fluidly connected to the filter for receiving the filtrate; and a second end fluidly connected to a capillary micropump for dispensing the filtrate to the capillary micropump; the capillary micropump being configured to receive the filtrate and to pump the filtrate through to the outlet. The plurality of capillaries may be substantially parallel relative to each other. The inlet may define a volume or reservoir and be configured to accept a sample. The inlet may define a volume of at least 50 μL, or at least 80 μL, or between about 50 μL to about 200 uL. The outlet may define a volume or reservoir and be configured to accept the filtrate. The outlet may define a series of outlet reservoirs.

The parameters of the filter, capillaries, and micropump may be selected for, or adapted to the sample to be passively separated. The filter may have a thickness suitable for the sample to be passively separated. The filter may have a thickness between about 1 μm to about 20 μm, or any specific value or specific range of values between about 1 μm to about 20 μm. The filter may have a thickness of about 9 μm. The filter pore size may be selected depending on the molecular size distribution, volume, and/or viscosity of the filtrate to be formed, and/or the molecular size of the analyte to be detected. The filter may comprise a filter membrane. The filter may have a pore size between about about 0.3 μm to about 10 μm; or any specific value or specific range of values between about 0.3 μm to about 10 μm. The filter may have a pore size about 0.6 μm. The width of the capillaries may be selected depending on the molecular size distribution, volume, and/or viscosity of the filtrate to be drawn into the capillaries. The capillaries may be spaced apart from each other at a distance about 1 to about 4 times their width, or about 3 times their width. The space between capillaries may be selected to increase the rate at which the filtrate moves through the capillaries. The capillaries may have a width between about 8 μm to about 20 μm; or any specific value or specific range of values between about 8 μm to about 20 μm. The capillaries may have a width of about 12 μm. The capillaries may be spaced apart from each other at a distance between about 8 μm to about 80 μm, or about 24 pm to about 60 μm; or any specific value or specific range of values between about 8 μm to about 80 μm. The capillaries may be spaced apart from each other at a distance of about 40 μm. The capillary micropump may have a width that increases and then decreases going between its first end and its second end. The capillary micropump may have an elliptical, half-hexagonal, or hexagonal shape. The capillary micropump may comprise a staggered array of substantially circular-, square-, rectangular-, or oval-shaped microstructures. The capillary micropump may function by creating capillary forces. Said capillary forces may facilitate movement of the filtrate from the filter to the outlet. The size and shape of the micropump may be selected to control the rate at which the filtrate moves through the device, to control the amount by which the microchannels are filled with filtrate, to control the volume of filtrate collected at the outlet, and/or to control flow resistance of the filtrate through the device.

The device may be fabricated using a microfluidic fabrication protocol and/or photolithography techniques. The filter, the parallel capillaries, and the capillary micropump of the device may be sandwiched between two layers. For example, the parallel capillaries and capillary micropump may be included in bottom layer, while a top layer is used to seal said microchannels. The device may be fabricated using polydimethylsiloxane (PDMS) as the fabricating material for each layer.

When in use, the device may function as follows. When a sample is introduced into the inlet of the device, the sample may come into contact with the filter, and the parallel capillaries and capillary micropump may facilitate withdrawing a filtrate from the filtrate side of the filter via capillary force, and may move or pump the filtrate through to the outlet. The filtrate may then collect within the outlet for collection or diversion. The device may be inverted or non-inverted. Non-inverted, as used herein, refers to the device being used upright. For example, the device may be used upright such that the inlet of the device—and therefore any sample introduced into it—is positioned substantially opposite the direction of gravity. Inverted, as used herein, refers to the device being upside down. For example, the device may be used upside down such that the inlet of the device—and therefore any sample introduced into it—is positioned substantially in the same direction of gravity. Using the device in an inverted position may permit heavier or more dense components (e.g., suspended solids) of a sample to settle opposite the filter, such that the sample is partially gravity-separated before coming into contact with the filter. This may help reduce or prevent clogging the filter pores, and may facilitate separating a larger amount of filtrate. This may also increase the filter's lifetime.

In another embodiment, the device comprises a body defining an inlet and an outlet, the inlet defining a volume or reservoir and configured to accept a sample, the body housing: a capillary microchannel, a capillary microwell, and a filter, the microchannel having a first end fluidly connected to the inlet for receiving the sample, and a second end fluidly connected to the microwell for dispensing the sample to the microwell, the microchannel configured to move the sample from the inlet to the microwell by capillary force, the microwell having a first end fluidly connected to the microchannel for receiving the sample, and a second end coupled to a filter, the microwell configured to move the sample from the microchannel to the filter by capillary force, the filter configured to filter the sample received from the microwell to produce a filtrate; and a plurality of capillaries and a capillary micropump, the plurality of capillaries configured to withdraw the filtrate from the filter by capillary force, each capillary having a first end fluidly connected to the filter for receiving the filtrate; and a second end fluidly connected to the capillary micropump for dispensing the filtrate to the capillary micropump; the capillary micropump being configured to receive the filtrate and to pump the filtrate through to the outlet. The plurality of capillaries may be substantially parallel relative to each other. The filter may be parallel to and/or vertically displaced from the microchannel. The inlet may define a volume of at least 50 μL, or at least 80 μL, or between about 50 μL to about 200 uL. The outlet may be parallel to and/or vertically displaced from the inlet. The outlet may define a volume or reservoir and be configured to accept the filtrate. The outlet may define a series of outlet reservoirs. The outlet may comprise a series of outlet reservoirs. As used herein, “vertically displaced” may refer to an element being positioned above and/or distanced from another element when the device is in a non-inverted position.

The parameters of the capillary microchannel, capillary microwell, filter, capillaries, and micropump may be selected for, or adapted to the sample to be passively separated. The width of the capillary microchannel may be selected depending on the volume, and/or viscosity of the sample to be drawn into the microchannel. The capillary microchannel may have a width between about 100 μm to about 1 mm; or any specific value or specific range of values between about 100 μm to about 1 mm. The capillary microchannel may have a width about 600 μm. The capillary microwell may be positioned substantially perpendicular to the microchannel. The width of the capillary microwell may be selected depending the molecular size distribution, volume, and/or viscosity of the sample to be drawn into the microwell. The microwell may have a width between about 1 mm to about 5 mm; or any specific value or specific range of values between about 1 mm to about 5 mm. The filter may have a thickness suitable for the sample to be passively separated. The filter may have a thickness between about 1 μm to about 20 μm, or any specific value or specific range of values between about 1 μm to about 20 μm. The filter may have a thickness of about 9 μm. The filter pore size may be selected depending on the molecular size, volume, and/or viscosity of the filtrate to be formed, and/or the molecular size of the analyte to be detected. The filter may comprise a filter membrane. The filter may have a pore size between about about 0.3 μm to about 10 μm. The filter may have a pore size about 0.6 μm. The width of the capillaries may be selected depending on the molecular size distribution, volume, and/or viscosity of the filtrate to be drawn into the capillaries. The capillaries may be spaced apart from each other at a distance about 1 to about 4 times their width, or about 3 times their width. The space between capillaries may be selected to increase the rate at which the filtrate moves through the capillaries. The capillaries may have a width between about 8 μm to about 20 μm; or any specific value or specific range of values between about 8 μm to about 20 μm. The capillaries may have a width of about 12 μm. The capillaries may be spaced apart from each other at a distance between about 8 μm to about 80 μm, or about 24 μm to about 60 μm; or any specific value or specific range of values between about 8 μm to about 80 μm. The capillaries may be spaced apart from each other at a distance of about 40 μm. The capillary micropump may have a width that increases and then decreases going between its first end and its second end. The capillary micropump may have an elliptical, half-hexagonal, or hexagonal shape. The capillary micropump may comprise a staggered array of substantially circular-, square-, rectangular-, or oval-shaped microstructures. The capillary micropump may function by creating capillary forces. Said capillary forces may facilitate movement of the filtrate from the filter to the outlet. The size and shape of the micropump may be selected to control the rate at which the filtrate moves through the device, to control the amount by which the microchannels are filled with filtrate, to control the volume of filtrate collected at the outlet, and/or to control flow resistance of the filtrate through the device.

The device may be fabricated using a microfluidic fabrication protocol and/or photolithography techniques. The device may integrate three different layers. The bottom-most layer (Layer 1) may contain the capillary microchannel. The middle layer (Layer 2) may contain the inlet for loading the sample and the microwell that connects to the plurality of capillaries and capillary micropump. The top layer (Layer 3) may contain said plurality of capillaries and the capillary micropump, as well as outlet reservoirs. The device may be fabricated using polydimethylsiloxane (PDMS) as the fabricating material for each layer.

When in use, the device may function as follows. When a sample is introduced into the inlet of the device, the sample may flow through the microchannel to the microwell by capillary force. The microwell, being substantially perpendicular to the microchannel, may act as a primary sample separation site due to built-in self-filtering. Within the microwell, automatic separation of the sample may occur because of the generation of a capillary force, or wettability gradient—the ability of the sample to maintain contact with the surface of the microwell, controlled by the balance between the intermolecular interactions, such as van der Waals interactions. Differences in hydrophilicity and/or active gravity at the center of the microwell may create a built-in self-filter, permitting heavier or more dense components (e.g., suspended solids) of a sample to settle opposite the filter, such that the sample is partially gravity-separated before coming into contact with the filter. This may help reduce or prevent clogging the filter pores, and may facilitate separating a larger amount of filtrate. This may also increase the filter's lifetime. The parallel capillaries and capillary micropump may then facilitate withdrawing a filtrate from the filtrate side of the filter via capillary force, and may move or pump the filtrate through to the outlet or outlet reservoirs. The filtrate may then collect within the outlet or outlet reservoirs for collection or diversion. In regard to this embodiment of the device, the device may used in a non-inverted configuration. In regard to this embodiment of the device, the device may not be used in an inverted configuration.

The sample introduced into the microfluidic passive sample separation (PSS) device may be a liquid sample. In one or more embodiments, the sample introduced into the microfluidic PSS device may comprise blood. The sample may comprise whole blood. The sample introduced into the microfluidic PSS device may comprise an aqueous solution. The sample may comprise urine. The sample may comprise seminal fluid. The sample may comprise vaginal fluid. The sample may comprise a tissue biopsy. The tissue may be human or animal. The tissue may be homogenized. The homogenized tissue may be uniformed and/or liquefied. The sample may comprise a marine or freshwater environment sample. The marine or freshwater environment sample may comprise a marine or freshwater water sample, a marine or freshwater plant, or marine or freshwater food. The marine or freshwater plant or food may be may be homogenized. The homogenized marine or freshwater plant or food may be may be uniformed and/or liquefied. The marine or freshwater food may be food sources within the marine or freshwater food chains. The sample may comprise phytoplankton. The phytoplankton may be homogenized. The homogenized phytoplankton may be uniformed and/or liquefied. The sample may comprise shellfish. The shellfish may be homogenized. The homogenized shellfish may be uniformed and/or liquefied. The sample may comprise food. The food may be homogenized. The homogenized food may be uniformed and/or liquefied. The food may be food suitable for consumption by living organisms, such as humans, animals, plants. The sample may comprise milk.

The sample may be introduced into the device without any pre-treatment steps or pre-processing steps, such as pre-filtering, pre-concentrating, or pre-separation steps. The sample may be introduced into the device in the same form or condition in which the sample was collected. Once introduced into the microfluidic PSS device, the sample may be separated into a liquid filtrate that has been separated from heavier or more dense components (e.g., suspended solids) of the sample.

The filtrate may comprise an analyte to be detected. The analyte may comprise a nucleic acid. The nucleic acid may comprise DNA. The nucleic acid may comprise circulating DNA (cDNA). The nucleic acid may comprise high-risk HPV (hr-HPV) circulating DNA (cDNA). The nucleic acid may comprise hr-HPV16 cDNA. The filtrate may comprise a protein. The protein may comprise an antibody. The protein may comprise an inflammatory marker. The inflammatory marker may comprise interleukins, cytokines, creative protein, pro-calcitonin, and/or pre-sepsin. The analyte may comprise biotoxins. The biotoxins may comprise domoic acid and/or okadaic acid. The analyte may comprise a cell, a bacterium, or a virus. The analyte may comprise a small molecule, such as a chemical compound. The analyte may comprise an antigen. The analyte may comprise a metabolite.

### Sensor

The microfluidic passive sample separation (PSS) device as described herein may further comprise a sensor. The sensor may be coupled to the to the outlet of the microfluidic PSS device. The sensor may be fluidly connected to the outlet of the device. The sensor may be used for detecting an analyte in the filtrate separated from the sample. The filtrate may move or be pumped from the microfluidic PSS device to the sensor by capillary force. The filtrate may move or be pumped from the microfluidic PSS device to the sensor by an applied suction force.

The sensor may be a microfluidic sensor. The sensor may be a miniaturized sensor. The sensor may comprise an electrochemical sensor, an optical sensor, a piezoelectric sensor, or a photodetector. The sensor may be selected based on the type of sample to be introduced into the microfluidic PSS device; the properties of the filtrate to be separated from the sample by the device; and/or the analyte to be detected in the filtrate. For example, the sensor may be selected based on the physical and/or chemical properties of the sample; the physical and/or chemical properties of the filtrate; and/or the physical and/or chemical properties of the analyte to be detected, or the concentration of the analyte to be detected.

In an embodiment, sensor may be an electrochemical sensor, an optical sensor, a piezoelectric sensor, or a photodetector suitable for detecting an analyte as described herein, such as a nucleic acid; DNA; circulating DNA (cDNA); high-risk HPV (hr-HPV) circulating DNA (cDNA); hr-HPV16 cDNA; a protein; an antibody; an inflammatory marker, such as interleukins, cytokines, creative protein, pro-calcitonin, and/or pre-sepsin; biotoxins, such as domoic acid and/or okadaic acid; a cell, a bacterium, or a virus; a small molecule, such as a chemical compound; an antigen; or a metabolite. In an embodiment, sensor may be an electrochemical sensor suitable for detecting an analyte as described herein, such as a nucleic acid; DNA; circulating DNA (cDNA); high-risk HPV (hr-HPV) circulating DNA (cDNA); hr-HPV16 cDNA; a protein; an antibody; an inflammatory marker, such as interleukins, cytokines, creative protein, pro-calcitonin, and/or pre-sepsin; biotoxins, such as domoic acid and/or okadaic acid; a cell, a bacterium, or a virus; a small molecule, such as a chemical compound; an antigen; or a metabolite.

In an embodiment, the sensor comprises an electrochemical sensor. The electrochemical sensor may comprise an electrode system. The electrode system may comprise a reference electrode, a counter electrode, and a working electrode (WE). The working electrode may comprise a carbon-based electrode, gold-based electrode or a graphene-based electrode. The working electrode may comprise a screen printed electrode (SPE). The working electrode may comprise a screen printed carbon electrode (SPCE). The working electrode may comprise a screen printed metal electrode (SPME). The working electrode may comprise a screen printed gold electrode (SPGE). The working electrode may comprise a screen printed silver electrode (SPSE). The working electrode may comprise a screen printed platinum electrode (SPPE).

The electrochemical sensor may comprise a probe immobilized on the working electrode (WE) for detecting an analyte in the filtrate. The probe be selected for, or adapted to detecting the analyte in the filtrate. The probe may be immobilized on the WE via bonding to a surface of the WE. The probe may be immobilized the WE via bonding to a coating on the WE. The probe may be immobilized on the WE via bonding to a carbon coating on the WE, such as covalent bonding to a graphene oxide coating. The probe may comprise complementary single stranded DNA (cssDNA).

The electrochemical sensor may further comprise a redox pair. The redox pair may be in solution. The redox pair may be housed within the sensor. The redox pair may be housed in the sensor, in contact with or as part of the electrode system. The redox pair may be introduced into the sensor ahead of use; for example, via an inlet. The redox pair may be introduced into the sensor such that is it in contact with, or forms part of the electrode system. The redox pair may comprise Ru(NH₃)₆³⁺+Fe(CN)₆³⁻ in which Ru(NH₃)₆³⁺ may be reduced to Ru(NH₃)₆²⁺ and Fe(CN)₆³⁻ catalyses the regeneration of Ru(NH₃)₆³⁺. The redox pair may comprise methylene blue, ferrocene, and/or dopamine.

### Integrated Detection Device—Microfludic Passive Sample Separation Device and Sensor

The microfluidic passive sample separation (PSS) device described herein may be coupled to, or in fluid communication with a sensor as described herein, forming an integrated detection device. So combined, the integrated device may be suitable for point-of-care (POC) use. So combined, the integrated device may be suitable for point-of-use (POU) detection. The integrated device may be sized to be portable by a single user. The integrated device may afford analyte detection with minimal to no sample pre-treatment or pre-processing, with minimal to no application of external forces to facilitate sample separation, and/or with minimal to no complex or cumbersome equipment.

In an embodiment, the integrated device may be used for detecting an analyte in a sample comprising introducing the sample into a microfluidic passive sample separation device, passing the sample through a filter of the microfluidic PSS device to form a filtrate; passing the filtrate through a plurality of capillaries of the microfluidic PSS device for drawing the filtrate through the filter by capillary force; pumping the filtrate by the capillary micropump of the microfluidic PSS device to an outlet; flowing the filtrate from the outlet to a sensor; contacting the filtrate with the sensor for a sufficient time and under suitable conditions to detect the analyte. In an embodiment, the integrated device may be used for detecting an analyte as described herein in a sample as described herein comprising introducing the sample into the microfluidic PSS device as described herein, passing the sample through the filter of the microfluidic PSS device to form a filtrate; passing the filtrate through the plurality of capillaries of the microfluidic PSS device for drawing the filtrate through the filter by capillary force; pumping the filtrate by the capillary micropump of the microfluidic PSS device to an outlet; flowing the filtrate from the outlet to a sensor as described herein; contacting the filtrate with the sensor for a sufficient time and under suitable conditions to detect the analyte.

The integrated device may be used for disease detection. The integrated device may be used to detect cancer. When the integrated device is used to detect cancer, the sample may comprise blood, whole blood, urine, seminal or vaginal fluid, or a tissue biopsy. When the integrated device is used to detect cancer, the analyte to be detected may comprise nucleic acids, DNA, circulating DNA, or a combination. When the integrated device is used to detect cancer, the cancer may be cervical cancer. When the cancer is cervical cancer, the analyte to be detected may comprise hr-HPV1 6 cDNA.

The integrated device may be used to detect sepsis. When the integrated device is used to detect sepsis, the sample may comprise blood, whole blood, urine, seminal or vaginal fluid, or a tissue biopsy. When the integrated device is used to detect sepsis, the analyte to be detected may comprise a protein, antibody, antigen, and/or metabolite. When the integrated device is used to detect sepsis, the analyte to be detected may comprise inflammatory markers. When the integrated device is used to detect sepsis, inflammatory markers may comprise proteins, such as interleukins, cytokines, creative protein, pro-calcitonin, and/or pre-sepsin.

The integrated device may be used to detect toxins. The toxins may comprise biotoxins. When the integrated device is used to detect toxins, the sample may comprise blood, whole blood, urine, a tissue biopsy, a marine or freshwater sample, a marine or freshwater plant, marine or freshwater food, phytoplankton, shellfish, or food for human, animal, or plant consumption. When the integrated device is used to detect toxins, the analyte to be detected may comprise domoic acid and/or okadaic acid.

The integrated device may be used to detect the presence of bacteria or viruses. When the integrated device is used to detect the presence of bacteria or viruses, the sample may comprise blood; whole blood; urine; seminal or vaginal fluid; a tissue biopsy; a marine or freshwater sample; a marine or freshwater plant; marine or freshwater food; phytoplankton; shellfish; or food for human, animal, or plant consumption. When the integrated device is used to detect toxins, the analyte to be detected may comprise a cell, a bacterium, and/or a virus.

In an embodiment, the integrated device may further comprise a treatment chamber, for pre-detection or post-separation treatment of the filtrate. Such treatment may be applied to permit detection, facilitate detection, or increase rate of detection. For example, if the analyte to be detected comprises a double-stranded nucleic acid (dsNA), the dsNA may need to be denatured before detection. The denaturing may occur by treating the dsNA with a base (for example, sodium hydroxide), or other denaturants, before detection. Such treatment may occur within a treatment chamber of the integrated device, the chamber being housed in the body of the microfluidic PSS device, within the sensor, or both.

In an embodiment, the integrated device may further comprise a heating element. The heating element may facilitate reducing viscosity of a filtrate to be analyzed. The heating element may facilitate denaturing an analyte to be detected. The heating element may facilitate increasing solubility of the filtrate; for example, to maintain analytes in solution for detection. The heating element may reduce incubating time for incubating the probe of a sensor with the analyte to be detected, which may decrease the time for detection. The heating element may be housed either in the body of the microfluidic PSS device, within the sensor, or both.

In an embodiment, using the integrated detection device may comprise signal amplification. Signal amplification may facilitate lowering the integrated detection device's limit of detection (LOD). Signal amplification may comprise increasing the concentration of the analyte to be detected. When the analyte comprises DNA, signal amplification may comprise DNA amplification. DNA amplification may involve polymerase chain reaction (PCR) to amplify the amount of DNA. To facilitate such amplification, a small chamber capable of performing the PCR may be added to the integrated device. Signal amplification may comprise increasing probe density in a sensor. When the sensor comprises an immobilized probe, increasing sites for immobilization may increase probe density, such as increases sites for bond formation to occur between a working electrode and probe. When the sensor comprises a working electrode, and said working electrode is to be coated in graphene-oxide (GO), selecting carboxylated GO, reduced GO, or fabricated GO nanocomposites may increase sites for bond formation, or increase surface area for reaction, thereby increasing probe density. Signal amplification may comprise increasing conductivity. When the working electrode is to be coated in graphene-oxide (GO), carboxylated GO, reduced GO, or fabricated GO nanocomposites may increase conductivity relative to graphene-oxide itself. Increasing conductivity may also comprise using a metal working electrode. The metal working electrode may comprise gold. Increasing conductivity may comprise using metal nanoparticles coupled to, or in conjunction with the working electrode. The metal nanoparticles may comprise gold nanoparticle.

As described herein, there is also provided:
1. A microfluidic passive sample separation device comprising: a body defining an inlet and an outlet, the body housing a filter for filtering a liquid sample deposited thereon and for producing a filtrate; a plurality of capillaries configured to withdraw the filtrate from the filter by capillary force, each capillary having a first end fluidly connected to the filter for receiving the filtrate; and a second end fluidly connected to a capillary micropump for dispensing the filtrate to the capillary micropump; the capillary micropump being configured to receive the filtrate and to pump the filtrate through the outlet.
2. The microfluidic passive sample separation device of embodiment 1, wherein the plurality of capillaries are substantially parallel.
3. The microfluidic passive sample separation device according to embodiment 1 or 2, wherein each capillary has a width and is spaced about three times the width from an adjacent capillary.
4. The microfluidic passive sample separation device according to any one of embodiments 1 to 3, wherein each capillary has a width of about 12 μm and is spaced about 40 μm from an adjacent capillary.
5. The microfluidic passive sample separation device of any one of embodiments 1 to 4, wherein the capillary micropump has at least a first end and a second end opposite the first end, and wherein the width of the micropump increases and then decreases between the first end and the second end.
6. The microfluidic passive filtration device of any one of embodiments 1 to 5, wherein the capillary micropump is an elliptical, half-hexagonal, or hexagonal capillary micropump.
7. The microfluidic passive sample separation device of any one of embodiments 1 to 6, wherein the capillary micropump is a hexagonal capillary micropump.
8. The microfluidic passive sample separation device of any one of embodiments 1 to 7, wherein the capillary micropump comprises a staggered array of substantially circular-, square-, rectangular-, or oval-shaped microstructures.
9. The microfluidic passive sample separation device of any one of embodiments 1 to 8, wherein the capillary micropump comprises a staggered array of substantially oval-shaped microstructures.
10. The microfluidic passive sample separation device according to any one of embodiments 1 to 9, wherein the liquid sample is about 80 μL to about 200 μL in volume.
11. The microfluidic passive sample separation device according to any one of embodiments 1 to 10, wherein the liquid sample is about 80 μL in volume.
12. The microfluidic passive sample separation device according to any one of embodiments 1 to 11, wherein the liquid sample comprises blood.
13. The microfluidic passive sample separation device according to any one of embodiments 1 to 12, wherein the liquid sample comprises whole blood.
14. The microfluidic passive sample separation device of embodiment 12 or 13, wherein the filtrate comprises plasma.
15. The microfluidic passive sample separation device according to any one of embodiments 1 to 14, wherein the analyte is a nucleic acid, protein, antibody, antigen, or metabolite.
16. The microfluidic passive sample separation device according to any one of embodiments 1 to 15, wherein the analyte is nucleic acid.
17. The microfluidic passive sample separation device according to any one of embodiments 1 to 16, wherein the analyte is DNA.
18. The microfluidic passive sample separation device according to any one of embodiments 1 to 17, wherein the analyte is circulating DNA.
19. The microfluidic passive filtration device according to any one of embodiments 1 to 18, wherein the analyte is hr-HPV16 cDNA.
20. The microfluidic passive filtration device according to any one of embodiments 1 to 19, wherein the filter is a filter membrane.
21. The microfluidic passive filtration device according to embodiment 20, wherein the filter membrane is sealed to the inlet.
22. The microfluidic passive filtration device according to embodiment 20 or 21, wherein the filter membrane has a thickness of about 9 μm.
23. The microfluidic passive filtration device according to any one of embodiments 20 to 22, wherein the filter membrane has a pore size of about 0.6 μm.
24. The microfluidic passive filtration device according to any one of embodiments 1 to 23, wherein the microfluidic passive filtration device is inverted.
25. The microfluidic passive filtration device according to any one of embodiments 1 to 24, wherein the microfluidic passive plasma separation device is suitable for point-of-care use and/or sized to be portable by a single user.
26. The microfluidic passive filtration device of embodiment 1 further comprising a sensor for detecting an analyte, the sensor being fluidly connected to the outlet.
27. A detection device for detecting an analyte in a liquid sample, the detection device comprising: a microfluidic passive sample separation device comprising: a body defining an inlet and an outlet, the body housing a filter for filtering a liquid sample deposited thereon and for producing a filtrate; a plurality of capillaries configured to withdraw the filtrate from the filter by capillary force, each capillary having a first end fluidly connected to the filter for receiving the filtrate; and a second end fluidly connected to a capillary micropump for dispensing the filtrate to the capillary micropump; the capillary micropump being configured to receive the filtrate and to pump the filtrate through the outlet; and an electrochemical biosensor, the electrochemical biosensor fluidly coupled to the microfluidic passive sample separation device for detecting the analyte in the filtrate.
28. The detection device according to embodiment 27, wherein the electrochemical biosensor comprises a working electrode having a surface comprising a probe configured to couple to, bind to, or hybridize with the analyte.
29. The detection device according to embodiment 28, wherein the working electrode is a carbon-based electrode, gold-based electrode or a graphene-based electrode.
30. The detection device according to embodiments 28 or 29, wherein the working electrode is a carbon electrode
31. The detection device according to any one of embodiments 28 to 30, wherein the working electrode is a screen-printed carbon electrode (SPCE).
32. The detection device according to any one of embodiments 28 to 31, wherein the probe is immobilized on the working electrode by drop-casting graphene oxide (GO) on the surface of the working electrode for forming a covalent bond between the probe and the GO.
33. The detection device according to embodiment 28 or 32, wherein the probe is cssDNA.
34. The detection device according to any one of embodiments 27 to 33, wherein the analyte is a nucleic acid, protein, antibody, antigen, or metabolite.
35. The detection device according to any one of embodiments 27 or 34, wherein the analyte is a nucleic acid.
36. The detection device according to any one of embodiments 27 or 35, wherein the analyte is DNA.
37. The detection device according to any one of embodiments 27 to 36, wherein the analyte is circulating DNA (cDNA).
38. The detection device according to any one of embodiments 27 to 37, wherein the analyte is hr-HPV16 cDNA.
39. The detection device according to any one of embodiments 27 to 38, wherein the liquid sample is about 80 μL to about 200 μL in volume.
40. The detection device according to any one of embodiments 27 to 39, wherein the liquid sample is about 80 μL in volume.
41. The detection device according to any one of embodiments 27 to 40, wherein the liquid sample comprises blood.
42. The detection device according to any one of embodiments 27 to 41, wherein the liquid sample comprises whole blood.
43. The detection device according to any one of embodiments 27 to 42, wherein the filtrate comprises plasma.
44. The detection device according to any one of embodiments 27 to 43, wherein the microfluidic passive filtration device is inverted.
45. The detection device according to any one of embodiments 27 to 44, wherein the detection device is suitable for point-of-care use and/or sized to be portable by a single user.
46. A method of separating a liquid sample, the method comprising: passing the liquid through a filter to form a filtrate; passing the filtrate through a plurality of capillaries for drawing the filtrate through the filter by capillary force; pumping the filtrate by capillary micropump for pumping the filtrate through the plurality of capillaries.
47. The method of embodiment 46, wherein the plurality of capillaries are substantially parallel.
48. The method of embodiment 46, wherein the micropump is a hexagonal capillary micropump.
49. The method of embodiment 46, wherein the liquid sample comprises blood.
50. The method of embodiment 46, wherein the liquid sample comprises whole blood.
51. The method according to any one of embodiments 46 to 50, wherein the analyte is a nucleic acid, protein, antibody, antigen, or metabolite.
52. The method according to embodiment 51, wherein the analyte is a nucleic acid.
53. The method according to embodiment 52, wherein the analyte is DNA.
54. The method according to embodiment 52, wherein the analyte is cDNA.
55. The method according to embodiment 52, wherein the analyte is hr-HPV 16 cDNA.
56. The method according to any one of embodiments 46 to 55, wherein the filter is a filter membrane.
57. The method according to any one of embodiments 46 to 56, further comprising contacting the analyte with an electrochemical biosensor probe for a sufficient time and under suitable conditions to detect the analyte.
58. A method of detecting an analyte in a liquid sample comprising: passing the liquid sample through a filter to form a filtrate; passing the filtrate through a plurality of capillaries for drawing the filtrate through the filter by capillary force; pumping the filtrate by capillary micropump to an electrochemical biosensor, the electrochemical biosensor comprising a probe configured to detect the analyte; contacting the filtrate with the probe for a sufficient time and under suitable conditions to detect the analyte; and detecting the presence of the analyte by electrochemical means.
59. The method of embodiment 58, wherein the electrochemical biosensor comprises a graphene-oxide screen-printed carbon electrode (GO-SPCE) probe chip.
60. The method according to embodiment 59, further comprising incubating the GO-SPCE probe chip at about 37° C. following the screening.
61. The method according to embodiment 60, wherein the GO-SPCE is incubated for about 10 to about 60 minutes.
62. The method according to embodiment 61, further comprising washing the incubated GO-SPCE probe chip with a buffer.
63. The method according to any one of embodiments 58 to 68, wherein the probe for the analyte is cssDNA.
64. The method according to any one of embodiments 58 to 63, wherein the graphene-oxide is drop casted on the surface of the SPCE.
65. The method of any one of embodiments 58 to 64, wherein the analyte is nucleic acid.
66. The method of any one of embodiments 58 to 66, wherein the analyte is DNA.
67. The method of any one of embodiments 58 to 67, wherein the analyte is cDNA.
68. The method according to any one of embodiments 58 to 67, wherein the analyte is hr-HPV 16 cDNA.
69. The method according to any one of embodiments 58 to 68, further comprising sedimenting the liquid sample prior to filtering the liquid sample.
70. The method according to any one of embodiments 58 to 69, wherein the liquid sample is inverted prior to filtering the liquid sample.
71. The method according to any one of embodiments 58 to 70 wherein the liquid sample comprises blood.
72. The method of any one of embodiments 58 to 71, wherein the liquid sample comprises whole blood.
73. A method of detecting an analyte in a whole blood sample comprising: passing the whole blood sample through a membrane filter to form a filtrate comprising plasma; passing the filtrate through a plurality of substantially parallel capillaries to isolate the plasma from the filtrate by capillary force; pumping the plasma by capillary micropump to an electrochemical biosensor, the electrochemical biosensor comprising a probe specific for the analyte; contacting the filtrate with the probe for a sufficient time and under suitable conditions to detect the analyte using the electrochemical biosensor; and detecting the presence of the analyte by electrochemical means.
74. The method of embodiment 73, wherein the electrochemical biosensor comprises a graphene-oxide screen-printed carbon electrode (GO-SPCE) probe chip.
75. The method of embodiment 73, further comprising sedimenting the whole blood sample prior to filtering the whole blood sample.
76. The method according to embodiment 74, further comprising incubating the GO-SPCE probe chip at about 37° C. following the screening.
77. The method according to embodiment 76, wherein the GO-SPCE is incubated for about 10 to about 60 minutes.
78. The method according to any one of embodiments 76 to 77, further comprising washing the incubated GO-SPCE probe chip with a buffer.
79. The method according to any one of embodiments 73 to 78, wherein the analyte is hr-HPV 16 cDNA.
80. The method according to any one of embodiments 73 to 79, wherein the probe for the analyte is cssDNA.
81. The method according to any one of embodiments 73 to 80, wherein the graphene-oxide is drop casted on the surface of the SPCE.
82. The method of any one of embodiments 75 to 81, wherein the analyte is nucleic acid.
83. The method of any one of embodiments 75 to 82, wherein the analyte is DNA.
84. The method of any one of embodiments 75 to 83, wherein the analyte is cDNA.
85. The method according to any one of embodiments 46 to 56, further comprising contacting the analyte with a sensor for a sufficient time and under suitable conditions to detect the analyte.

As described herein, there is also provided:
1. A microfluidic passive sample separation device comprising
   a body defining an inlet and an outlet, the inlet defining a volume and configured to accept a liquid sample,
   the body housing:
      a capillary microchannel, a capillary microwell, and a filter,
         the microchannel having a first end fluidly connected to the inlet for receiving the liquid sample, and a second end fluidly connected to the microwell for dispensing the liquid sample to the microwell, the microchannel configured to move the liquid sample from the inlet to the microwell by capillary force,
         the microwell having a first end fluidly connected to the microchannel for receiving the liquid sample, and a second end coupled to a filter, the microwell configured to move the liquid sample from the microchannel to the filter by capillary force,
      the filter configured to filter the liquid sample received from the microwell to produce a filtrate; and
      a plurality of capillaries and a capillary micropump,
         the plurality of capillaries configured to withdraw the filtrate from the filter by capillary force, each capillary having a first end fluidly connected to the filter for receiving the filtrate; and a second end fluidly connected to the capillary micropump for dispensing the filtrate to the capillary micropump;
         the capillary micropump being configured to receive the filtrate and to pump the filtrate through to the outlet.
2. The device of embodiment 1, wherein the filter is: vertically displaced relative to the microchannel; and/or parallel to the microchannel.
3. The device of embodiment 1 or 2, wherein the outlet is: vertically displaced relative to the inlet; and/or parallel to the inlet.
4. The device of any one of embodiments 1 to 3, wherein the outlet comprises a series of outlet reservoirs.
5. The device of any one of embodiments 1 to 4, wherein the capillary microchannel has a width between about 100 μm to about 1 mm.
6. The device of any one of embodiments 1 to 5, wherein microwell has a width between about 1 mm to about 5 mm.
7. The device of any one of embodiments 1 to 6, wherein the plurality of capillaries are substantially parallel; and wherein optionally each capillary has a width of about 8 μm to about 20 μm and is spaced about 8 μm to about 80 μm from an adjacent capillary; or each capillary has a width of about 12 μm and is spaced about 40 μm from an adjacent capillary.
8. The device of any one of embodiments 1 to 7, wherein the capillary micropump has at least a first end and a second end opposite the first end, and wherein the width of the micropump increases and then decreases between the first end and the second end.
9. The device of any one of embodiments 1 to 8, wherein the capillary micropump comprises: an elliptical, half-hexagonal, or hexagonal capillary micropump; and/or a staggered array of substantially circular-, square-, rectangular-, or oval-shaped microstructures.
10. The device of any one of embodiments 1 to 9, wherein the capillary micropump comprises: a hexagonal capillary micropump; and/or a staggered array of substantially oval-shaped microstructures.
11. The device of any one of embodiments 1 to 10, wherein the filter is a filter membrane.
12. The device of embodiment 11, wherein the filter membrane has: a thickness of about 1 μm to about 20 μm, or about 9 μm; and/or a pore size of about 0.3 μm to about 10 μm; or about 0.6 μm.
13. The device of any one of embodiments 1 to 12, wherein the microfluidic passive filtration device is non-inverted.
14. The device of any one of embodiments 1 to 13, wherein the inlet defines a volume of at least 50 μL, or at least 80 μL, or between about 50 μL to about 200 uL.
9. The device of any one of embodiments 1 to 14, wherein the liquid sample comprises blood; whole blood; urine; seminal fluid; vaginal fluid; tissue; an aqueous solution; marine or freshwater water; a marine or freshwater plant; marine or freshwater food; phytoplankton; shellfish; food; or milk; or a combination thereof.
16. The device of any one of embodiments 1 to 15, wherein the filtrate comprises an analyte, and the analyte optionally comprises a nucleic acid, DNA, circulating DNA, high-risk HPV circulating DNA, high-risk HPV16 circulating DNA, a protein, an antibody, an antigen, a metabolite; a toxin; a biotoxin, such as domoic acid or okadaic acid; a small molecule; a cell; a bacterium; a virus; or an inflammatory marker, such as interleukins, cytokines, creative protein, pro-calcitonin, or pre-sepsin.
17. The device of any one of embodiments 1 to 16, wherein the microfluidic passive plasma separation device is suitable for point-of-care use and/or sized to be portable by a single user.
18. The device of any one of embodiments 1 to 17, further comprising a sensor for detecting an analyte, the sensor being fluidly connected to the outlet, and the sensor optionally comprising an electrochemical sensor, an optical sensor, a piezoelectric sensor, or a photodetector.

Various compositions, systems and methods will be described herein to provide an example of at least one embodiment of the claimed subject matter. No embodiment described herein limits any claimed subject matter and any claimed subject matter may cover compositions, systems, devices and methods that differ from those described below. The claimed subject matter is not limited to compositions, systems, devices and methods having all of the features of any one composition, system, device or method described below or to features common to multiple or all of the compositions, systems, devices and methods described below. It is possible that any composition, system, device or method described below is not an embodiment of any claimed subject matter. Any subject matter that is disclosed in a composition, system, device or method described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

In the claims, as well as in the specification above, all transitional phrases such as “comprising,” “including,” “carrying,” “having,” “containing,” “involving,” “holding,” “formed from”, “composed of,” and the like are to be understood to be open-ended, i.e., to mean including but not limited to.

Only the transitional phrases “consisting of” and “consisting essentially of” shall be closed or semi-closed transitional phrases, respectively.

The following examples are intended to illustrate certain exemplary embodiments of the present disclosure. However, the scope of the present disclosure is not limited to the following examples.

### EXAMPLES

### Example 1. An Integrated Microfluidic Electrochemical Assay for Cervical Cancer Detection at Point-of-Care Testing

### Summary

Cervical cancer (CC) is a major health care problem in low- and middle-income countries, necessitating the development of low-cost and easy-to-use assays for CC detection at point-of-care settings. An exemplary integrated microfluidic electrochemical assay for CC detection, named IMEAC, is described below for identifying CC circulating DNA in the whole blood samples. The exemplary IMEAC consists of two main modules: a plasma separator device that isolates plasma from whole blood with high purity and without the need for any external forces, which is connected to a graphene oxide-based electrochemical biosensor that uses specific probe molecules for detection of CC circulating DNA molecules. The performance of the individual modules were characterized, and show that the integrated assay can advantageously be utilized for target DNA detection in whole blood samples, thus potentially transforming CC detection and screening at remote locations.

### Introduction

Cervical cancer (CC) is a global problem, standing as the fourth most common cancer among women worldwide. Over half a million women developed CC and over a quarter of million women died from CC globally in 2018. The CC burden is substantially higher in low- and middle-income countries (LMIC), with nine in ten CC-associated deaths occurring in LMIC. CC, if detected early, is highly treatable. CC screening and treatment at early stages cost less than $500 USD and has 90% survivability, whereas treating the invasive CC costs about $5,000 USD with only 15% survivability. Limited access to complex laboratory equipment and professionals for sample processing and analysis in LMIC considerably contributes to the higher CC burden in these regions.

The etiological cause of CC is persistent infection with high-risk strains of human papilloma virus (hr-HPV) where long-lasting infection with hr-HPV16 and 18 contributes to most of CC. The cancerous nature of hr-HPV16 and 18 attributes to two of its genes, E6 and E7 genes, encoding E6 and E7 oncoproteins. Lasting infection with hr-HPV16 and 18 leads to integration of E6 and E7 genes into host genome, leading to progression of the cervical cells to cancerous lesions. Current common practices in developed countries are pap smear, histological test performed by gynaecologists to check cell abnormalities, and polymerase chain reaction (PCR) test, looking at the presence of hr-HPV in the cervical samples. Pap smear tests are not easily accessible in LMIC, and hr-HPV PCR tests cannot predict the likelihood of developing CC.

Recent studies suggest that hr-HPV circulating DNA (cDNA) is found in blood plasma and can act as a marker for CC prognosis. In addition, hr-HPV cDNA levels in blood posttreatment might indicate the cancer recurrence. The hr-HPV cDNA dissemination mechanism is not yet completely understood and is suggested that dying cancer cells might shed the hr-HPV cDNA into the blood stream.

Detection and analysis of cDNA in whole blood is an appealing approach for non-invasive cancer diagnosis and monitoring. Electrochemical-based assays for DNA detection have already demonstrated promising alternatives for conventional PCR test and clinical sample analysis. These chip-based assays offer several potential advantages, including rapid response, easy automation, high sensitivity, amenability to multiplexed detection, capability of integration with sample processing, and not-relying on costly laboratory instrumentation, enabling their usage in LMIC. Electrochemical techniques have been employed for hr-HPV DNA detection in cervical samples. A gold-coated carbon-based screen printed genosensor was developed for detection of hr-HPV18 in spiked cervical samples. To recognize hr-HPV18 DNA, a complementary single stranded DNA (cssDNA) was used as the probe. In another work, modified screen-printed carbon electrodes (SPCEs) with peptide nucleic acid capture probe were employed to detect amplified hr-HPV16 DNA in HPV16-positive cell culture samples. The previously reported electrochemical biosensors measured the hr-HPV DNA in cervical samples. An electrochemical assay to detect hr-HPV cDNA in extracted plasma samples is desirable.

The existence of large numbers of red blood cells (RBCs) and white blood cells (WBCs) in whole blood samples, can significantly interfere with the biosensor performance and can limit the use for potential clinical applications. For successful detection of biomolecules from whole blood, it can be important to separate plasma. On-chip sample preparation methods have taken advantage of microfluidic devices containing networks of tiny channels and chambers through which minute amounts of reagents move. The unique advantages of microfluidic systems, which can make them a superior candidate for portable point-of-care (POC) diagnostic devices, are the capability of measuring from small volumes of fluidics and integrating with downstream analysis. Ideally, sample processing and target biomarker detection steps can be carried out using a single miniaturized platform for POC setting.

An integration of a flow driven microfluidic device for plasma isolation and depletion of background blood cells with downstream analysis, such as electrochemically detection of cDNA, has the potential to transform the current CC diagnosis in LMIC locations into a POC setting.

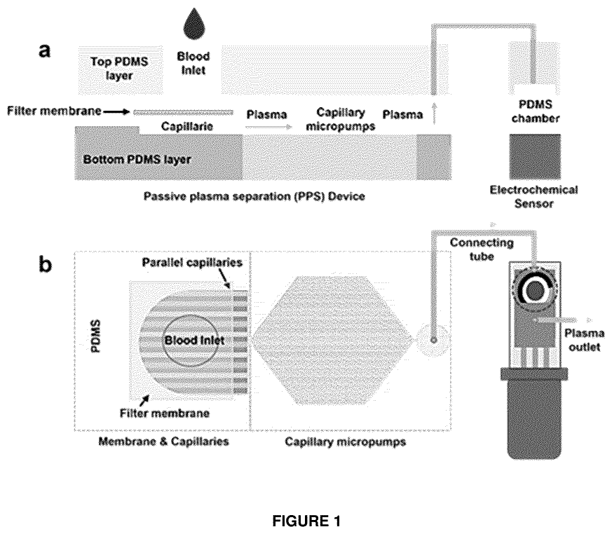

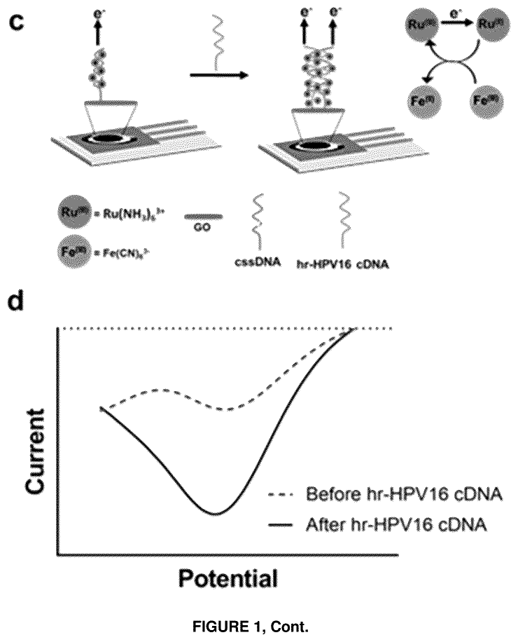

Herein described is an integrated detection device, an embodiment of which include a microfluidic electrochemical assay which may be used for diagnosis of cancers such as cervical cancer (which may thus be referred to as IMEAC), that can be automated to detect hr-HPV16 cDNA from extracted plasma samples. The IMEAC combines two main modules to achieve hr-HPV cDNA detection (FIG. 1): a passive sample separator (PSS) microfluidic device which may be used for separating and isolating plasma (otherwise referred to as a passive plasma separator (PPS)) without a need for applying external force, and sensor that comprises a graphene oxide (GO) based-electrochemical biosensor that employs cssDNA probe for specific hr-HPV 16 cDNA recognition. The PPS device can collect approximately 22 μL of plasma from 160 μL of whole blood without any dilution in 10 min. An advantage of the PPS device is that the separation is automatic, and plasma reaches the outlet without the application of any pressure force due to the capillary action [29,33]. The electrochemical biosensor integrates low-cost SPCEs where drop-casting of GO is employed for cssDNA probe immobilization. The IMEAC can permit detection of hr-HPV 16 cDNA directly from extracted plasma sample that can be used for POC screening of CC at remote locations.

### Results and Discussion

### IMEAC Device Principle

Without wishing to be bound by theory, outlined below are general principals believed to contribute to the functionality and advantages of the integrated detection device of this embodiment.

The schematic of IMEAC which integrates the PPS and a sensor comprising the graphene oxide-screen-printed carbon electrode (GO-SPCE) for hr-HPV 16 cDNA detection is shown in FIG. 1. To enable passive plasma separation for POC testing, a microfluidic device was designed with parallel capillaries and capillary micropump with a filter membrane at the inlet (FIG. 1a and b). The PPS design was modified through experimentation to achieve an optimal plasma collection volume (yield) with high purity (about 99%) from whole blood. The integration of filter membrane with parallel capillaries and capillary micropump allows for autonomous withdrawing of plasma from the membrane surface when a few drops of whole blood was placed at the inlet via capillary forces. Since no external force was applied, the lysis of RBCs was minimal using the PPS, which can be one of the major challenges for active plasma separation. Upon collection, plasma containing circulating viral DNA can then be directed to a senor for electrochemical detection. The electrochemical biosensor used was based on a three electrode system comprising a silver/silver chloride (Ag/AgCl) reference electrode, carbon counter electrode, and carbon working electrode (WE) integrated in commercially available screen printed carbon electrode. To immobilize the cssDNA probe, GO was drop casted on the surface of the carbon WE, where the purchased amine-modified probe formed covalent amide binding with carboxyl groups presented on the GO surface. A sensitive Ru(NH₃)₆³⁺+Fe(CN)₆³⁻ electrochemical system was employed for cDNA detection in which Ru(NH₃)₆³⁺ is reduced to Ru(NH₃)₆²⁺ and Fe(CN)₆³⁻(Fe(III) to Fe(II)) catalyses the regeneration of Ru(NH₃)₆³⁺ (FIG. 1c). Ru(NH₃)₆³⁺ is electrostatically attracted to the nucleic acid on the surface of WE in amounts proportional to number of negative charges presented on the electrode's surface. Upon incubation of plasma samples containing hr-HPV16 cDNA, the capture probe hybridizes with the target DNA leading to an increase in the number of negative charges on WE surface, thus accumulation of more Ru(NH₃)₆³⁺ and higher electrochemical signal (FIG. 1d).

### Design and Fabrication of Microfluidic PPS Device

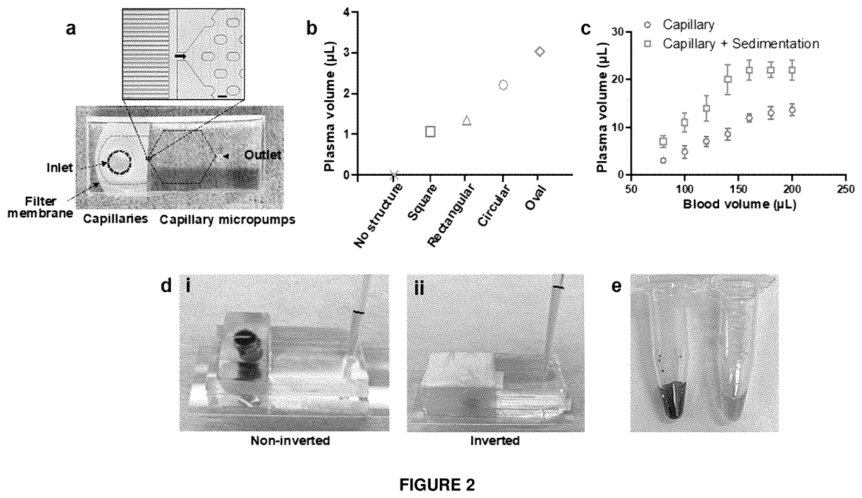

The PPS device, as an example of the microfluidic PSS device described herein, was fabricated using a microfluidic fabrication protocol with polydimethylsiloxane (PDMS). The PPS device comprised three main components: a filter membrane at the inlet, parallel capillaries, and capillary micropump which are sandwiched between two PDMS layers (FIG. 2a). The parallel capillaries and capillary micropump were included in bottom PDMS layer while a top PDMS layer used to seal the micro-channels. A 9 μm thick filter membrane with average pore size of 0.6 μm was employed for filtration of RBCs and WBCs. The thickness of the membrane was suitable for bonding between two PDMS layers using oxygen plasma treatment.

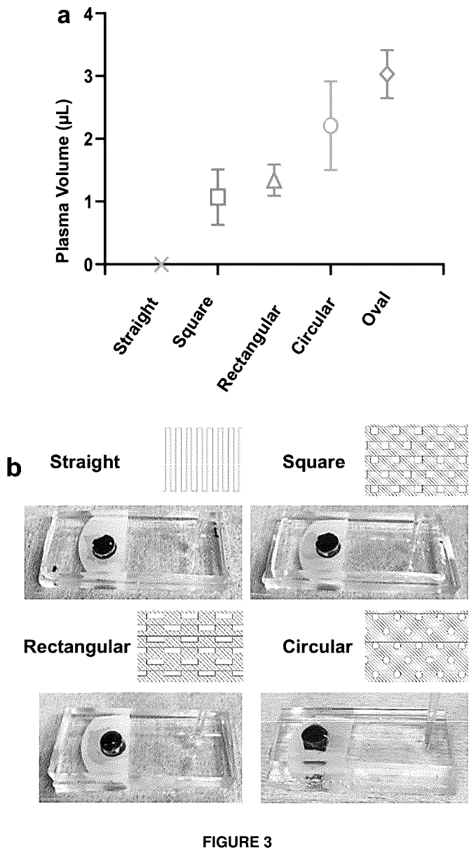

The parallel capillaries and capillary micropump facilitated withdrawing the plasma from the filtrate side of the membrane due to the capillary force. Different designs of microcapillaries were tested to reduce the dead-end volume. Capillary channels with a width of 12 μm and 40 μm apart from each other was found optimum for plasma collection with a dead volume of approximately 9 μL (FIG. 2a). Capillary micropump with different designs (square, rectangular, circular, and oval structures) were tested to achieve a sufficient channel filling. An array of microstructures was placed in such a manner that the micropump has a comparatively lower flow resistance due to the large number of parallel flow paths, thus facilitating the plasma collection. The micropump performance was compared based on collected plasma volume in 10 min using the same inlet volume of whole blood (80 μL) (FIG. 2b and FIG. 3a and b) and it was found that the plasma collection volume at the outlet was maximum using oval shaped micropump. All of the filtration experiments were conducted with this micropump design. A comparison of the collected plasma from inverted and non-inverted devices led to the observation that there was difference in collected plasma volume for the same blood volume at the inlet (FIG. 2c). In the case of a non-inverted device, it was observed that blood cells started settling down on top of the membrane surface due to gravity, which could result in the clogging of membrane pores and complete cease of filtration (FIG. 2d-i). For the inverted device, the sedimentation of blood cells assisted the settlement of plasma on top and permitted the capillary force to transfer a higher volume of plasma (FIG. 2d-ii). The inverted technique was found to increase the membrane lifetime and plasma volume at the outlet compared to the non-inverted technique. In addition, it was found that inverting the device upon wrapping the inlet hole with a parafilm (FIG. 2d-ii) increased the plasma collection volume by sedimentation. This experiment also showed that the capacity of the PPS device is to collect 22 μL of plasma from 160 μL whole blood, and any further increase in the inlet blood volume (200 μL) may not increase the collected plasma volume (FIG. 2c). FIG. 2e shows the collected plasma (about 200 μL) using the PPS device compared to the same volume of whole blood.

TABLE 1
Comparison of PPS performance with other recent studies.

| Recent | | | | | | |
|---|---|---|---|---|---|---|
| reported | | | | | Fil- | |
| works on | Plasma | Blood | | | tration | |
| plasma | volume | volume | Purity | Yield | time | |
| separation | (μL) | (μL) | (%) | (%) | (min) | Remark |
| Centrifuge, | 137 | 400 | 99 | 34 | 10 | WB-45% |
| Control | | | | | | haematocrit |
| Ref ^{[30]} | 0.45 | 5 | 99 | 9 | 15 | DB-Capillary, |
| | | | | | | Passive |
| Ref ^{[32]} | 12 | 100 | — | 12 | <10 | WB (45%)- |
| | | | | | | Capillary, Pump |
| Ref ^{[25]} | 132 | 400 | — | 33 | 5 | WB (45%)- |
| | | | | | | Pipetting, Large |
| | | | | | | dead volume |
| Ref ^{[38]} | 43 | 243 | 100 | 18 | 10 | DB (15%)-Vacuum |
| | | | | | | pump |
| PPS device | 22 | 160 | 99 | 25 | 10 | WB (45%)- |
| | | | | | | Capillary, Passive |

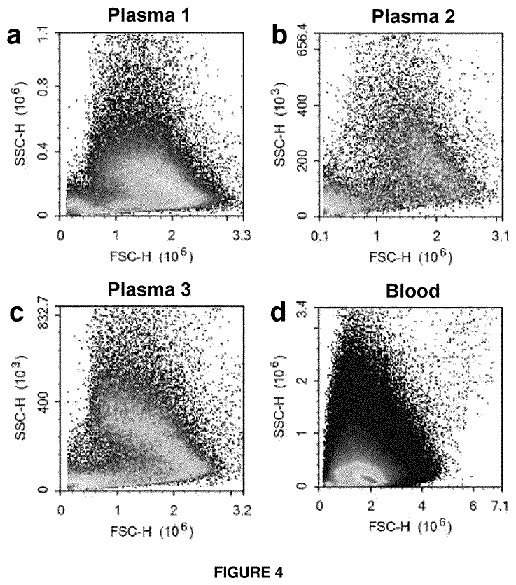

The purity and yield were also calculated to benchmark the microfluidic PPS device performance with previously reported works (Table 1, above). Purity of the filtered plasma was estimated by counting the number of blood cells in the inlet blood and outlet plasma using flow cytometry (FIG. 4; Table 2) and the yield was calculated based on dividing the collected plasma volume by the blood volume. The purity of the PPS device was comparable to other studies where some of these works used diluted blood (DB), prepared a blood sample with desired haematocrit level as compared to whole blood (WB), and used active force to extract plasma.

TABLE 2
Flow cytometry data for purity calculations

| Sample | No of Total Cell | Filtered Cell | Purity |
|---|---|---|---|
| Plasma 1 | 80546400 | 5271960000 | 98.4952 |
| Plasma 2 | 21234400 | 5331272000 | 99.6033 |
| Plasma 3 | 46886400 | 5305620000 | 99.124 |
| Blood | 5352506400 | | |

### Design and Characterization of Electrochemical Biosensor

Prior to testing with extracted plasma sample, the capability of the electrochemical biosensor was tested for detection of hr-HPV16 cDNA spiked into buffer solution. An experiment to immobilize the cssDNA probes on GO surface was performed using the amine-coupling technique through which the amine modified probe molecules form covalent amide bonding with carboxyl groups presented on GO surface.

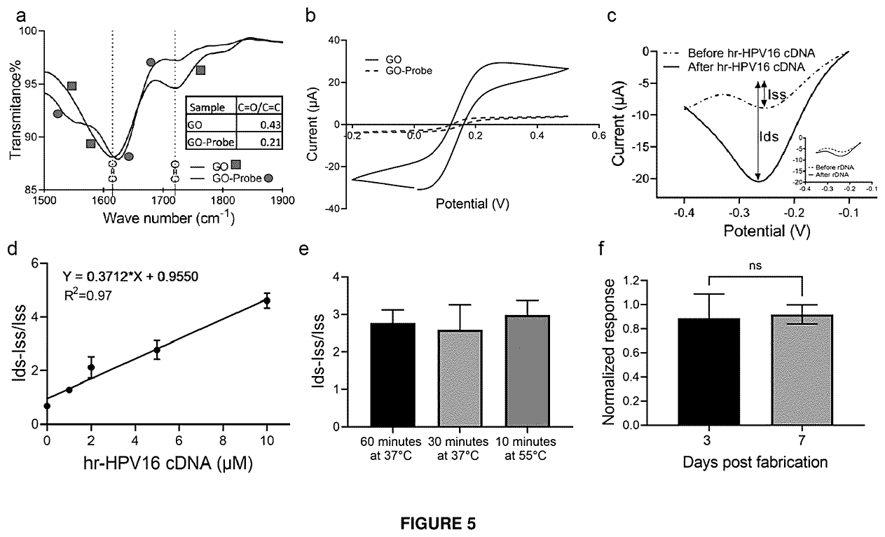

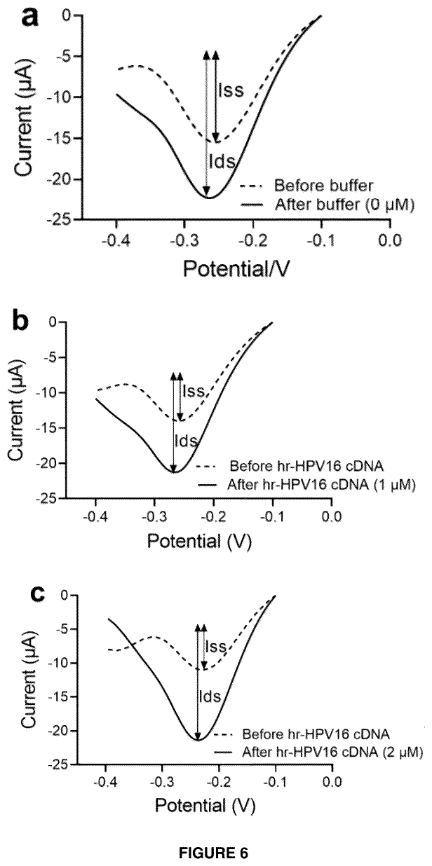

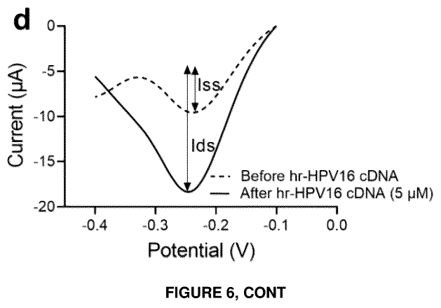

Fourier transform infrared (FTIR) was performed to study probe immobilization on the GO surface (FIG. 5a). The peaks at 1720 cm^{μ-1} and 1610 cm⁻¹ of the FTIR spectrum of GO display the C═O and C═C stretching vibrations, respectively. The spectrum of GO before probe immobilization (square line) showed a strong C═O peak, which significantly decreased in intensity after probe immobilization (circle line), indicating that the C═O bonds of GO were broken to form the covalent linkage with amine modified probes. To quantify the decrease in C═O bond after probe immobilization, the ratio of C═O peak intensity to C═C peak intensity (a constant value) was measured. As evident in Fig. the ratio of C═O/C═C for GO has been reduced to more than half of its initial value after probe immobilization. The probe immobilization was further studied by performing cyclic voltammetry (CV) measurement of GO samples grafted with cssDNA probe in Fe(CN)₆^{3-/2-} solution. Since both nucleic acid probe and Fe(CN)₆^{3-/2-} contain negative charges, a repletion occurs between them leading to a decrease in the redox current. Thus, a lower CV peak is expected in the presence of probe molecules. As shown in FIG. 5b, the redox peak current was significantly lower for the GO sample after probe immobilization, confirming that the probe was functionalized. To measure the amount of probe molecules, GO was functionalized with the cssDNA probe, and methylene blue (MB) was used to measure the probe density. This approach employed the interaction between MB and guanine bases of a DNA strand to quantify the number of immobilized oligonucleotides. The quantified average density of probe on GO was 6.8 μmol/cm². Having successfully functionalized GO with cssDNA probe molecules, the capability of the electrochemical biosensor for detection of hr-HPV16 cDNA spiked in buffer solution was studied. GO-SPCE grafted with cssDNA probes were incubated with different concentrations of target DNA (1 μM- 10 μM). The change in the current upon hybridization with target DNA was assessed by performing differential pulse voltammograms (DPV) and quantitating peak currents before (dotted line) and after (solid line) hybridization (FIG. 5c and FIG. 6a-d). In this approach, the Ru(NH₃)₆³⁺ was electrostatically attracted to the negatively charged probe molecules or hybridized probe-target nucleic acid strands on the electrode surface. Ru(NH₃)₆³⁺ was then reduced to Ru(NH₃)₆²⁺ when triggered at its reduction potential (−0.3 to −0.2 V). The difference between the obtained peak currents before and after target hybridization was used as the measure to correlate with target concentration. Adding a non-specific target did not appear to generate a significant change in DPV current after incubation (FIG. 5c-inset), demonstrating the specificity of the sensor for hr-HPV16 cDNA detection. FIG. 5d shows the sensor's electrochemical response to different concentrations of spiked hr-HPV16 cDNA in buffer solution which is fitted into a linear curve with R²=0.97. As displayed in FIG. 5d, in the presence of higher concentrations of hr-HPV16 cDNA, more Ru(NH₃)₆³⁺ was electrostatically bound to WE, leading to increased changes in the DPV measurement. To make the operating time shorter, experiments were conducted at different temperature. It was found that the incubation time can be shortened from 1 hr at 37° C. to 10 minutes at 55° C. for hr-HPV16 cDNA (FIG. 5e). A miniaturized, low-cost heating element can be integrated within the assay to increase the temperature, which may facilitate effective and rapid detection. The assay and the electrochemical biosensors immobilized with the cssDNA probes were stored for 3 or 7 days to assess stability. No significant change in the signal measurement was observed (FIG. 5f), demonstrating that the sensor can be stable at least for 7 days.

### Detection of Cervical Cancer'S Biomarker in Extracted Plasma Sample

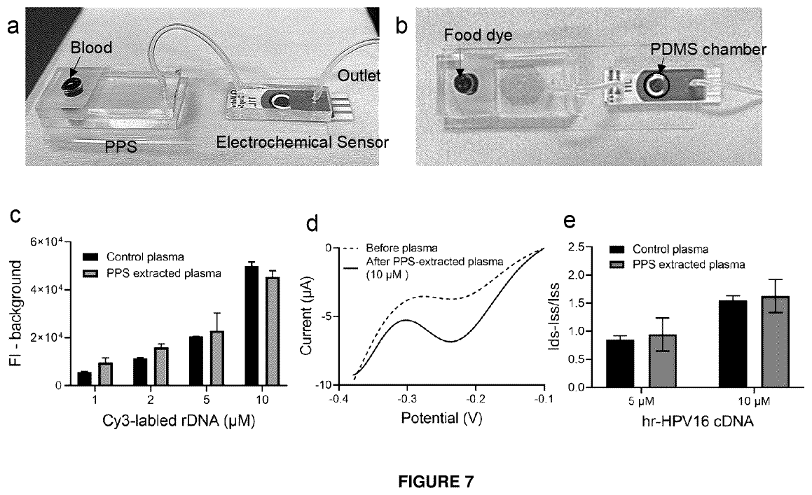

Having demonstrated and optimized the PPS performance for plasma separation and the electrochemical biosensor for hr-HPV16 cDNA detection, the IMEAC was subsequently fabricated (FIG. 7). The PPS outlet was connected via a tube to a PDMS chamber covering the electrodes of modified GO-SPCE. FIG. 7a shows the side view of the integrated platform and FIG. 7b shows a demonstration of the assay with food dye. Upon blood loading, the plasma containing the target DNA was isolated and directed towards the electrochemical biosensor modified with cssDNA probe molecules via withdrawing using a syringe attached to the outlet. Efficiency of the PPS device to recover the target DNA was studied. A whole blood sample was spiked with different concentrations of a random DNA molecule (rDNA, with approximately similar size to hr-HPV16 cDNA) labeled with Cy3 fluorophore and loaded into the PPS device. The fluorescence intensity measurement of the collected plasma from the PPS device which interrogates Cy3 was very close to the control plasma samples spiked with the rDNA molecule, demonstrating that on average 130% of DNA was recovered (FIG. 7c). Subsequently, whole blood samples were spiked with two concentrations of the hr-HPV16 cDNA (5 μM and 10 μM) and loaded into IMEAC device for plasma separation and DNA detection. Before and after target hybridization, DPV measurement was performed (FIG. 7d). As shown in FIG. 7e, the change in the peak current of DPV measurement was higher for 10 OA spiked sample compared to the 5 μM one. Control experiments where the centrifuged collected plasma was spiked with 5 μM and 10 μM target DNA were also performed. IMEAC measurements matched well with the ones obtained from the control experiment. The lower measurements obtained from plasma compared to the buffer experiments could be attributed to the background molecules present in plasma that block electron transfer. It should be noted that in real samples, the hr-HPV cDNA presents in double strand (ds) form and should be denatured before sensing. This can be achieved by mixing the isolated plasma with 1 molar NaOH³⁹. A chamber can be integrated into the IMEAC that contains dried NaOH for this purpose. Incubating dsDNA with 1 molar NaOH can denature dsDNA in 5 minutes without affecting its hybridization capability.

### Material and Methods

### Material

Polydimethylsiloxane (PDMS) elastomer (Sylgard 184) was obtained from Dow Corning, and SU8-3050 and SU8-developer from Kayaku Advanced Material. Whole blood sample was purchased from BiolVT (US). Filter membrane (Average pore size, 0.6 μm) was obtained from Sterlitech. N-(3-Dimethylaminopropyl)-N′-ethylcarbodiimide hydrochloride (EDC, Sigma Aldrich, 98%), N-Hydroxysuccinimide (NHS, Sigma Aldrich, 98%), 4-Morpholineethanesulfonic acid, 2-(N-Morpholino)ethanesulfonic acid (MES, Sigma Aldrich, >99%), Potassium Hexacyanoferrate (III) (Fe(CN)₆³⁻, Sigma Aldrich, 99%), Potassium Hexacyanoferrate (II) (Fe(CN)₆²⁻, Sigma Aldrich, 99%), Hexaammineruythenium (III) chloride (Ru(NH₃)₆³⁺, Sigma Aldrich, 98%), Magnesium chloride hexahydrate (MgCl₂, Fisher scientific), Sodium phosphate (Sigma Aldrich, 96%, NaPO₄), Phosphate buffer solution (PBS, Sigma Aldrich), Sodium chloride (Millipore Sigma), Methylene blue (MB, Sigma Aldrich), Screen printed carbon electrodes (SPCE, DRP-11L-U75, Metrohm, Canada), All synthetic nucleic acids (sequences provided in Table 3) were purchased from Integrated DNA Technologies (IDT). Graphene Oxide (GO) was provided by KPA (https://graphenika.com/).

TABLE 3
Nucleic acid sequences

| Nucleic acid | |
|---|---|
| strand | Sequence |
| hr-HPV 16 | GGGCTCTGTCCGGTTCTGCTTGTCCA/3AmMO/ |
| cssDNA (Probe) | (SEQ ID NO. 1) |
| hr-HPV 16 cDNA | TGGACAAGCAGAACCGGACAGAGCCC |
| (Target) | (SEQ ID NO. 2) |
| Cy3-rDNA | /5Cy3/GGTGGTGGGGGGGGTTGGTAGGGTGTC |
| | TTC (SEQ ID NO. 3) |

### Method

### Fabrication of the PPS Device

A photolithography technique was used to fabricate a microfluidic PPS device using PDMS. First a mold was prepared using a 4″ Si wafer, which was cleaned with piranha to remove organic impurities. The wafer was then HF treated to remove any native oxide layer. A negative photoresist SU 8 3025 was spin coated at 4000 rpm to deposit a layer of 25 μm following the datasheet provided by supplier Kayaku Advanced Material. After that the photoresist was baked (95° C. for 10 min) and exposed to the designed pattern and developed in the developer solution. When the mold was ready, PDMS (10:1) was poured into the master mold and cured at 80° C. for 2 hours. To make the PPS device, a commercial filter was bonded at the inlet using oxygen plasma treatment. Then the PPS device was then bonded to another PDMS layer by oxygen plasma to create a micro-channel after drilling inlet and outlet. A 6.3 mm diameter inlet hole and 1.2 mm diameter outlet hole were punched on the top PDMS layer before bonding the two PDMS layers. More layers of PDMS at the inlet were introduced to increase the blood volume at inlet.

### Plasma Collection using a PPS Device

Human whole blood was used for all the experiments. To examine the efficacy of the PPS device, undiluted whole blood was directly injected into the inlet hole. The inlet whole size was kept constant for all experiments, and the height of the PDMS layers was varied to add more volume of blood. Plasma was collected from the outlet by pipetting for characterization.

### GO-SPCE Fabrication and Probe Modification

Before use, the SPCE chips were rinsed with acetone, isopropanol, and deionized water followed by a pre-treatment protocol. The pre-treatment step included five CV cycles from +0.5 to −1.5 V in 0.1 M HCl followed by two CV cycles from 0 to +2 V in phosphate buffer solution (0.1 M PBS, pH 7) at a scan rate of 50 mV/s. The SPCEs were subsequently washed with ultrapure water (MiliQ) and dried. 10 μL of GO (from 1 mg/mL stock) was then drop-casted on the SPCE working electrode (WE) surface and dried overnight. To immobilize the probe on GO-SPCE, the surface was first activated by incubating the WE with 50 μL of an equimolar solution of EDC-NHS (100 mM) in MES buffer (100 mM, PH 6.8) overnight. The EDC-NHS was washed quickly, and the chips were incubated with 20 μl of 15 μM amine-modified probe (hr-HPV16 cssDNA) in probe buffer (20 mM magnesium chloride, 25 mM sodium chloride, 25 mM PBS) in a humid chamber for 2 hours. After the probe incubation time, the chips were extensively washed with 1XPBS to remove any unattached probes.

### FTIR Characterization

The chemical composition and functional groups of blank GO-SPEC and GO-SPCE functionalized with the probe molecules were characterized using FTIR characterization (Tensor 27 FTIR, Bruker).

### Probe Density Measurement

The probe density was measured using MB reporter described in Hu et al (Biosens. Bioelectron., 2011, 26, 4355-436141), incorporated herein by reference. Briefly, the modified chips were incubated with 20 μM MB (in B-R Buffer +20 mM sodium chloride, pH=6) in a beaker for 5 min, while stirring (to improve accumulation of MB). The electrodes were then washed with 1XPBS and subjected to CV scanning from 0.12 to −0.4 with scan rate of 50 mV/s in B-R buffer +20 mM sodium chloride. Before MB accumulation, a similar CV scan was performed on chips in B-R buffer that was taken as the background and subtracted from the final scan. To obtain the charge quantity of accumulated MB, the difference in the area under the curve for MB reduction was measured. The probe density was subsequently calculated using the formula N=Q/(n [symbol] N_{A}), where N represents the mol quantity of MB, Q is the electrical charge quantity of MB reduction, n is the number of electrons participating in the reaction (2 in this process), e the electric charge quantity of one electron (1.6×10⁻¹⁹ C), and N_{A} is the Avogadro's number (6.02×10²³ mol⁻¹). Since the hr-HPV16 cssDNA probe contained 8 guanine bases, the calculated N was then normalized by 8. To measure the surface area of WE, a diameter of 4 mm was used (provided by Metrohm).

### Target Hybridization

For buffer experiments, the desired concentration of hr-HPV16 cDNA (target) was diluted in target buffer (25 mM sodium chloride and 1.6 mM sodium phosphate) and incubated with the GO-SPCE chip functionalized with probe molecules for 1 hour at 37° C. in a humid chamber. The GO-SPCE chips were washed with 1X PBS after the target hybridization. For plasma experiments, the collected plasma was directly incubated with the chips with no dilution and the surface of modified GO-SPCE was blocked using 0.05% BSA (20 minutes incubation followed by PBS wash) prior to target hybridization.

### Electrochemical Measurement

The electrochemical measurements were performed by either CHI (CH Instruments) or Palmsens4 potentiostat. CV scans were obtained in 10 mM Fe(CN)₆²⁻/Fe(CN)₆³⁻ solution using 50 mV/s scan rate. Differential pulse voltammetry (DPV) scans were performed in 100 μM Ru(NH₃)₆³⁺+25 mM sodium phosphate (pH 7), 25 mM sodium chloride, and 4 mM Fe(CN)₆³⁻. DPV signals before and after hybridization were measured using an increment potential of 4 mV, pulse amplitude of 50 mV, pulse width of 50 ms, and a pulse period of 50 ms (translating to 8 mv/s scan rate). The GO-SPCE chips were rinsed with 1X PBS after the DPV scans.

### Stability Experiment

For stability tests the GO-SPCE chips immobilized with the cssDNA were refrigerated in the probe buffer for 3 and 7 days. The sensor was then incubated with 5 μM of the target DNA and the electrochemical measurement were then performed as explained above. The sensor response was normalized to the measurement obtained from a freshly made device.

### Study of Different Incubation Conditions

Prepared GO-SPCE-Probe chips were subjected to DPV using the mentioned set-up. The chips were then incubated with 5 μM of target at three different conditions: i) 37° C. and 1 hr incubation time, ii) 37° C. and 30 minutes incubation time, and iii) 55° C. and 10 minutes incubation time. The chips were washed with 1X PBS and again subjected to DPV. The biosensor response (Ids-Iss/Iss) was calculated and reported in FIG. 5d.

### IMEAC Fabrication

The electrochemical sensor was integrated to the PPS device to generate the IMEAC. At first, a thin layer of PDMS (1-2 mm thickness) was deposited on top of the unmodified SPCE sensor to facilitate PDMS-PDMS bonding using oxygen plasma. Next, a circular PDMS chamber covering the electrode was cut and PDMS was removed from that area. The electrochemical sensor was then modified with GO and the cssDNA probe molecule was immobilized as described above. Next, the modified SPCE was bonded to a PDMS chamber with a straight microfluidic channel. The chamber was aligned with the electrodes. The PDMS chamber inlet was then connected to the outlet of the PPS device via tubing and the PDMS chamber outlet was connected to a tube to enable withdrawing solution out of the IMEAC in one single step (FIG. 7b).

### hr-HPV16 DNA Detection using IMEAC Device

200 μL of whole blood sample spiked with 5 μM and 10 μM concentrations of hr-H PV16 cDNA was loaded at the inlet chamber of the PPS device. Once the outlet of the PPS device was filled with plasma, using a syringe at the outlet of the PDMS chamber of the electrochemical sensor, the collected plasma was directed to the sensor. The plasma containing the hr-HPV16 DNA was incubated with the electrochemical biosensor immobilized with the capture DNA probes for 1 hr at 37C° and the detection was conducted as explained in the electrochemical measurement section.

### Plasma Collection using Centrifuge

The control plasma was collected by centrifuging blood at 2000 rcf for 10 minutes. The collected plasma was then spiked with target to make the final concentrations of 5 μM and 10 μM and tested using the GO-SPCE-Probe chips.

### Study of the Efficiency of the PPS Device for DNA Recovery

To examine the presence of spiked nucleic acid in the collected plasma from the PPS device, 200 μL of blood was spiked with different concentrations of a Cy3-labled 30-bp random DNA (rDNA). The blood was then injected into the PPS device and the isolated plasma was collected. The collected plasma was diluted 1:20 in 1XPBS and the fluorescent intensity was measured using BioTek Synergy H1 plate reader (excitation: 530 nm, peak emission: 570 nm). As control, different concentrations of rDNA were spiked into isolated plasma and the fluorescence signal was measured. The fluorescent intensity of PPS-extracted plasma was then normalized to the fluorescent intensity of control plasma spiked with same concentrations of Cy3-labled rDNA and reported as the recovery percentage (130%).

### Non-Binding Conclusions

Early diagnosis and detection of CC biomarkers are crucial for successful cancer treatment. In this study, a low-cost and integrated detection device—microfluidic electrochemical assay, named IMEAC when used for CC detection, was designed that enabled detection of hr-HPV16 cDNA in extracted plasma sample with little to no sample processing or need for external costly equipment. Capillary force along with sedimentation was used to isolate plasma while the detection was achieved via GO modified SPCE immobilized with cssDNA probe molecules that detect hr-HPV16 cDNA target. The IMEA can be potentially employed in identifying other biomarkers of CC, such as hr-HPV18 cDNA from plasma by using suitable probe molecules, thus enabling multiplexed measurement. An advantage of the IMEAC is that it can be non-invasive and can use blood for CC detection. The passive filtration strategy avoids the use of conventional “gold standard” centrifugation methods, which require access to external equipment and materials. Thus, the IMEAC device can be used as an assay that generates plasma from whole blood for multiplexed detection of CC biomarkers at the POC testing setting. The concentration range of hr-HPV cDNA in plasma of cervical cancer patients is 1,099 copies/mL. The limit of detection (LOD) was found to be 0.48 μM which translated to −109 copies/mL. Therefore, to detect the physiological range of hr-HPV cDNA in plasma, either a DNA amplification step can be integrated, or the sensitivity of the electrochemical biosensor can be improved to enable rare target DNA detection. DNA amplification can be integrated similar to the previously reported microfluidic devices that enable performing polymerase chain reaction (PCR) on chip [42-44]. Incorporation of carboxylated GO [35], reduced GO [45,46], or fabricating GO nanocomposites can increase the probe density, thus improving the sensitivity. In the assay, vials of washing buffer and detection reagent (Ru(NH₃)₆³⁺+Fe(CN)₆³⁻) along with disposable pipettes can be supplemented for performing washing steps as well as the detection step.

### References for Example 1

[25] X. Su , J. Zhang , D. Zhang , Y. Wang , M. Chen , Z. Weng , J. Wang , J. Zeng , Y. Zhang , S. Zhang , S. Ge , J. Zhang and N. Xia. , Micromachines, 2020, 11, 352
[29] M. S. Maria, T. S. Chandra and A. K. Sen, Microfluid. Nanofluidics, 2017, 21, 1-21.
[30] M. Sneha Maria, P. E. Rakesh, T. S. Chandra and A. K. Sen, Biomicrofluidics, 2016, 10, 1-15.
[32] A. Homsy, P. D. van der Wal, W. Doll, R. Schaller, S. Korsatko, M. Ratzer, M. Ellmerer, T. R. Pieber, A. Nicol and N. F. de Rooij, Biomicrofluidics, 2012, 6, 1-9.
[33] M. Zimmermann, H. Schmid, P. Hunziker and E. Delamarche, Lab Chip, 2007, 7, 119-125.
[35] S. Yang, F. Zhang, Z. Wang and Q. Liang, Biosens. Bioelectron., 2018, 112, 186-192.
[38] Y. Lee, D. M. Kim, Z. Li, D. E. Kim and S. J. Kim, Lab Chip, 2018, 18, 915-922.
[42] J. Khandurina, T. E. McKnight, S. C. Jacobson, L. C. Waters, R. S. Foote and J. M. Ramsey, Anal. Chem., 2000, 72, 2995-3000.
[43] T. H. Fang, N. Ramalingam, D. Xian-Dui, T. S. Ngin, Z. Xianting, A. T. Lai Kuan, E. Y. Peng Huat and G. Hai-Qing, Biosens. Bioelectron., 2009, 24, 2131-2136.
[44] Z. Li, Y. Bai, M. You, J. Hu, C. Yao, L. Cao and F. Xu, Biosens. Bioelectron., 2021, 177, 112952.
[45] B. Li, G. Pan, N. D. Avent, R. B. Lowry, T. E. Madgett and P. L. Waines, Biosens. Bioelectron., 2015, 72, 313-319.
[46] C. Chan, J. Shi, Y. Fan and M. Yang, Sensors Actuators, B Chem., 2017, 251, 927-933.
[47] A. Singh, G. Sinsinbar, M. Choudhary, V. Kumar, R. Pasricha, H. N. Verma, S. P. Singh and K. Arora, Sensors Actuators, B Chem., 2013, 185, 675-684.

### Example 2. A Microfluidic Plasma Separation Device for Biomarker Detection in Whole Blood

### Introduction

A whole blood testing is commonly used for detecting disease related agents, such as protein biomarkers. Blood testing usually requires plasma extraction from whole blood to remove background blood cells that can interfere with signal measurement. This often needs expensive equipment (e.g., a centrifuge) and skilled personnel. To enable POC testing, separating plasma in-situ is desirable to leave all plasma protein properties intact for sensitive and reliable sensing of biomarkers without the interference of blood cells [22]. In addition, centrifugation results in RBC lysis which may produce nonspecific signal [22].

As described herein, the microfluidic PSS or PPS device comprises a body housing a filter membrane at the inlet which separates plasma and a capillary design, where a combination of parallel capillaries and capillary micropump facilitates the withdrawal of plasma from the permeate side and drives it towards the outlet. The PSS or PPS device could be integrated with an electrochemical biosensor for cervical cancer biomarker detection. The embodiment of the PSS device described in Example 1 (PPS) could be used with an inversion technique to facilitate sedimentation of blood cells away from the filter, with larger dead volume (otherwise referred to as lost volume) provided the inlet was sealed (e.g., with parafilm). For Point-of-care (POC) application, it was considered that technical modification of the PSS device to avoid the inversion technique may be desired, so that the modified microfluidic PSS or PPS device integrated with a sensor may further leverage the possibility of low-cost, specific, and multiplexed sensing in remote locations.

### Materials

Polydimethylsiloxane (PDMS) elastomer (Sylgard 184) was obtained from Dow Corning, and SU8-3050 and SU8-3025 photoresist and developer solution were purchased from Kayaku Advanced Material. Human whole blood sample was purchased from BiolVT (US). The filter membrane (average pore size, 0.6 μm) was obtained from Sterlitech. All chemicals such as phosphate buffer solution (PBS, Sigma Aldrich),3-Aminopropyltriethoxysilane (APTES) were purchased from Sigma Aldrich or Fisher Scientific

### PS-V2 Device Design and Fabrication

The embodiment of the microfluidic PSS device as described in Example 1 was modified to operate without need of inversion and with a lower dead-end volume. The embodiment of the device as described in Example 1 is referred to as PS-V1 in this example, and the embodiment of the device as described in Example 2 is referred to as PS-V2 (Example 2).

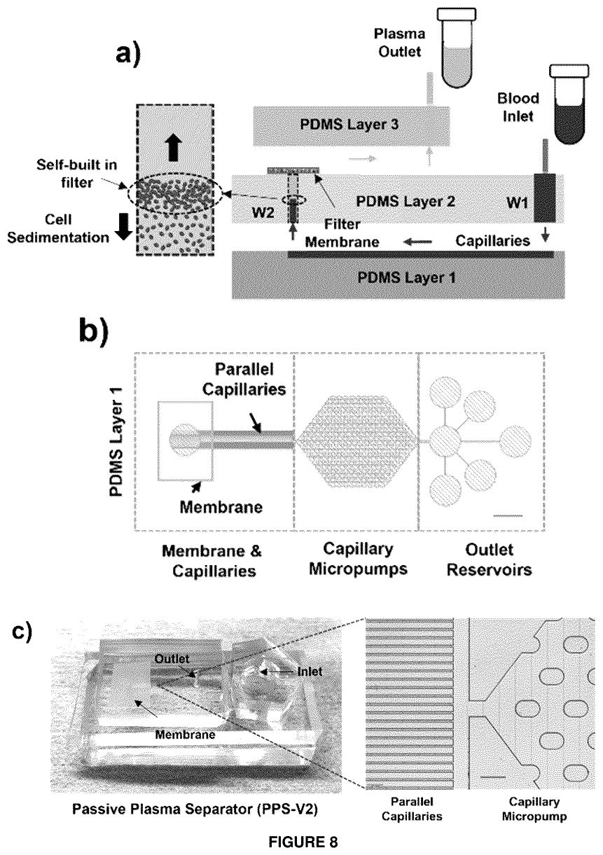

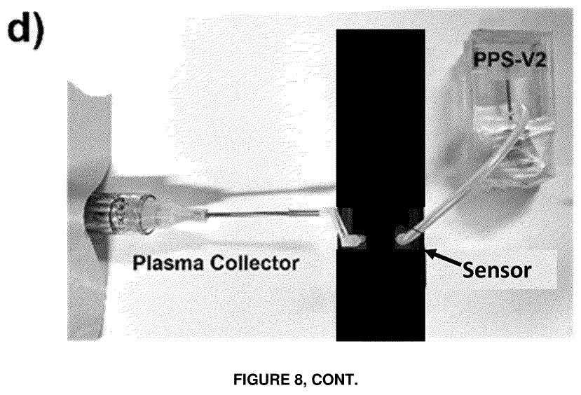

The PS-V2 device was fabricated with PDMS using microfabrication methods and photolithography techniques [32]. The device integrates three PDMS layers from bottom to top with microcapillaries and a 9 um thick membrane in between middle and the top layer (FIG. 8a). The bottom layer (PDMS Layer 1) contains a straight capillary microchannel (W=600 μm, H=25 μm) which can help transport blood from the inlet to the end of capillary in less than one minute. The middle PDMS layer (PDMS Layer 2>5 mm thickness) consists of a 6.3 mm inlet well (W1) for loading a sample, such as a blood sample and a 1.2 mm outlet well (W2) that connects to the top fluidic microchannels. The top layer (PDMS Layer 3) consists of the parallel capillaries and the capillary micropump adopted from PS-V1 for plasma collection, and outlet reservoirs, such as plasma outlet reservoirs [32]. The dead volume of the PS-V2 device was reduced (−4.50 vs 90 for original PPS device) for both parallel capillaries and capillary micropump, and additional outlet reservoirs were added to store the accumulated plasma (FIG. 8b)[32]. When blood is loaded at W1, it flows through capillary and reaches W2. The well W2 serves as an anchor site for the primary plasma separation due to a self-built in filter (FIG. 8a). At W2, automatic plasma separation can occur because of the generation of a wettability gradient; the ability of a liquid to maintain contact with a solid surface which is controlled by the balance between the intermolecular interactions, such as van der Waals interactions [26]. The plasma cannot reach the centre of W2 due to the cylindrical layout and small dimension [26], causing the centre to remain less hydrophilic and forming a wettability gradient (FIG. 8a) [26]. The difference in hydrophilicity and active gravity at the centre of W2 creates a self-built in filter made of accumulated blood cells for primary plasma separation [26]. A 9 μm thick filter membrane with an average pore size of 0.6 μm was cut into small rectangular pieces (7 mm×5 mm) and bonded between middle (PDMS Layer 2) and the top Layer (PDMS layer 3) using plasma bonding (FIG. 8c) to improve blood cell depletion. APTES was used to strengthen the bonding at membrane-PDMS interfaces before plasma bonding [33]. The APTES treatment was performed by treating the porous membrane with O₂ plasma for 1 min, then it was immersed in a 5% (v/v) 3-aminopropyltriethoxysilane (APTES) solution at 80° C. for 20 min before it was dried [33, 34]. Next, the porous membrane and the PDMS top and bottom layers were bonded together using O₂ plasma for 30 seconds. All PDMS layers were bonded together using oxygen plasma-activated bonding in such a manner that the inlet and outlet hole openings in the bottom layer, middle layer, and top channels were aligned (FIG. 8c). Next, the devices were annealed at 70° C. for at least 15 minutes to ensure proper sealing. Additional PDMS layers at the inlet and outlet can be added to incorporate tubing for integration (FIG. 8d).

The design and dimensions of different layers of PS-V2 were selected to achieve the desired plasma separation [32]. A small volume (80-200 μl) of whole blood was loaded at the inlet. Upon loading, the blood reached to the outlet of the bottom layer due to capillary force, which was observed to occur in less than one minute. The wettability gradient in the middle layer's well and sedimentation resulted in primary isolation of plasma in less than 10 min and the presence of the filter membrane improved the separation with a relatively high purity (−86%). The plasma was then collected from the outlet reservoirs using a pipette or syringe. As with the PS-V1 embodiments, the PS-V2 device was also designed to easily connected to a sensor inlet via tubing for in-situ and POC detection (FIG. 8d).

### Results and Discussion

### Studying Plasma Extraction using PS-V2 Device

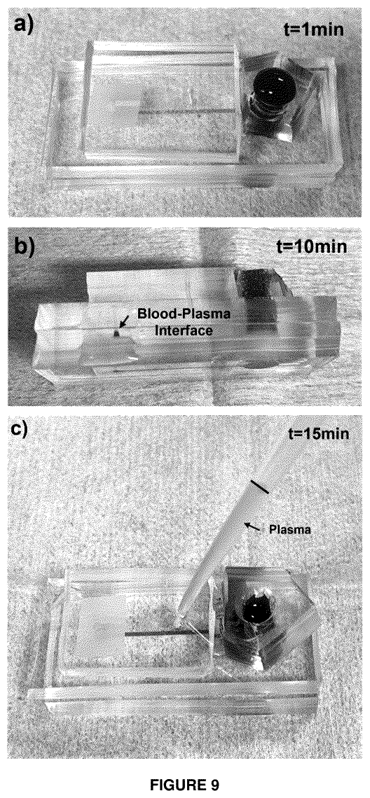

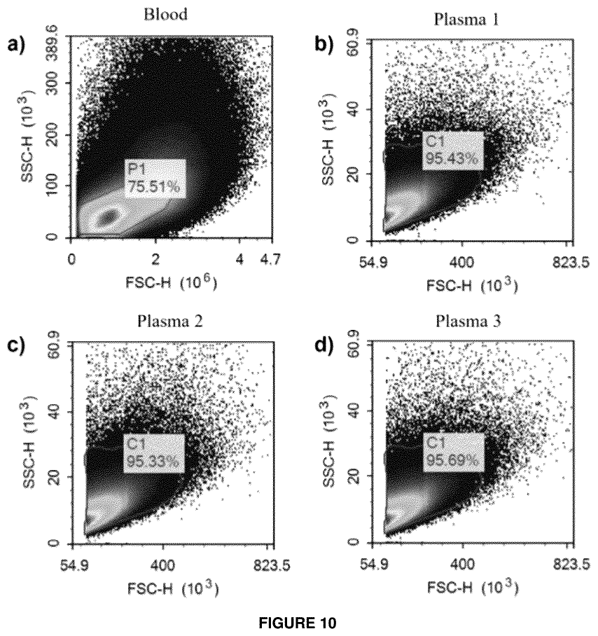

Plasma collection using the PS-V2 was characterized. The PS-V2 device was designed such that it did not use the inversion technique; filter membrane pores may be less clogged as fewer blood cells can reach the membrane pores; and it may be more easily integrated with a sensor. When used with whole blood, the PS-V2 inlet filled with whole blood (80-200 μl), and blood flowed through the bottom capillary microchannel in approximately 1 min (FIG. 9a). Next, the automated plasma separation occurred in the microwell due to sedimentation and wettability gradient in approximately 10 min (FIG. 9b). A built-in self-filter in the well can form such that only plasma with fewer blood cells move to the top microchannels [27]. The formation of this built-in self-filter may put less stress on the membrane surface and avoid leakage, which may facilitate high purity filtration (FIG. 9c and FIG. 10). The clear plasma was collected in a pipette tip after 15 min of blood loading (FIG. 9c). The purity of the filtered plasma was calculated using flow cytometry and was assessed by counting the number of blood cells in the inlet blood and outlet plasma (FIG. 10). The yield was obtained by dividing the collected plasma volume using the system by the plasma volume using centrifugation [32]. A purity of ˜86% and a yield of ˜25% were demonstrated using PS-V2 device, as described in further detail below.

Flow cytometry analysis was performed for the collected plasma from the PS-V2 device outlet. At first, 22 μl of plasma was collected form the outlet of three PS-V2 devices to prepare three plasma samples from the same whole blood sample. All collected plasma samples were diluted (1:10 dilution ratio) in PBS buffer solution containing 1×phosphate-buffered saline (PBS), 0.05% sodium azide (NaN₃) and 2 g/L bovine serum albumin (BSA) to avoid cell adhesion to the channel walls. Four samples of 300 μL each diluted blood and plasma samples were prepared for the flow cytometry analysis and analyzed right after filtration. All cells were counted through flow cytometry (NovoCyte Flow Cytometer, Agilent Technologies, San Diego, CA, USA) and FIG. 10a-d is generated for unfiltered blood and filtered plasma samples. Blood cells were assessed from the dot plot representation and subsequently gated through forward (FSC) and side scatter (SSC) and set to a linear scale (FIG. 10). From FIG. 10(a), the overlap of RBC and WBC populations in forward and side scatter were counted together as blood cells and same gating were applied to plasma samples for comparison.

The purity (P) of the PS-V2 device was calculated using the following equation (1):

$$P = \frac{\# of filtered cell}{\# of total cell} \% \qquad ( 1 )$$

The number of filtered cells were calculated from the flow cytometry data as shown in Table 4 below.

TABLE 4
Flow cytometry data for the blood and filtered plasma samples using PPS-V2
device.

| Sample | Count | Abs. Count | % Parent | Median X | Median Y | Filtered cell | Purity |
|---|---|---|---|---|---|---|---|
| Blood | 2712630 | 1176720800 | 75.51 | 953543 | 44518 | | |
| Plasma 1 | 805437 | 164663600 | 95.43 | 150234 | 9770 | 1012057200 | 86.00657012 |
| Plasma 2 | 764390 | 156168200 | 95.33 | 150863 | 9690 | 1020552600 | 86.72852558 |
| Plasma 3 | 783474 | 160118600 | 95.69 | 151731 | 9644 | 1016602200 | 86.39281298 |

Per Table 4, the recorded and averaged purity was 86.37. The yield (Y) of was calculated using the formula below (equation 2).

$$Y = \frac{Collected volume of plasma using device described herein}{Collected volume of plasma for ideal condition} \% \qquad ( 2 )$$

### Non-Binding Conclusions

This example presents a low-cost, portable, and integrated microfluidic plasma separation device that can be integrated with a sensor, such as a biosensor to detect analytes such as biomolecules in whole blood sample without the need for pre-processing of sample or external costly equipment. The PS-V2 microfluidic device employed capillary force along with sedimentation and wettability gradient to isolate plasma and facilitate biomarker sensing. The PS-V2 device allowed for collection of 22 μI of plasma from whole blood (160 μl) in approximately 15 minutes. The PS-V2 device can be integrated to various sensors or diagnostic assays for various applications at POC testing [32]. An advantage of the PS-V2 is the use of whole blood for biomarkers detection with little or no sample pre-processing; it is easy-to-fabricate, portable, and disposable; and it uses a low reagent sample volume. The PS-V2 may enable POC testing at remote locations where costly laboratory equipment and skilled personnel are not accessible.

All references cited in this document are incorporated herein by reference in their entirety.

The above-described embodiments are intended to be examples only. Alterations, modifications and variations can be effected to the particular embodiments by those of skill in the art without departing from the scope of the disclosure, which is defined solely by the claims appended hereto.

### References for Example 2

[22] O. S. Jacqueline Jatschka, Andre Dathe, Andrea Csaki, Wolfgang Fritzsche, Sens. Biosensing Res., 2016, 7, 62-70.
[26] M. S. Maria, P. E. Rakesh, T. S. Chandra and A. K. Sen, Sci. Rep., 2017, 7, 1-12.
[27] M. Sneha Maria, P. E. Rakesh, T. S. Chandra and A. K. Sen, Biomicrofluidics, 2016, 10, 1-15.
[32] F. Keyvani, N. Debnath, M. Ayman Saleh and M. Poudineh, Nanoscale, DOI:10.1039/d1nr08252c.
[33] Y. Lee, D. M. Kim, Z. Li, D. E. Kim and S. J. Kim, Lab Chip, 2018, 18, 915-922.
[34] M. Agostini, G. Greco and M. Cecchini, APL Mater, DOI:10.1063/1.5070136.

1. A microfluidic passive sample separation device comprising:
a body defining an inlet and an outlet, the body housing
a filter for filtering a liquid sample in contact therewith and for producing a filtrate; and
a plurality of capillaries configured to withdraw the filtrate from the filter by capillary force, each capillary having
a first end fluidly connected to the filter for receiving the filtrate; and
a second end fluidly connected to a capillary micropump for dispensing the filtrate to the capillary micropump;
the capillary micropump being configured to receive the filtrate and to pump the filtrate to the outlet.

2. The microfluidic passive sample separation device of claim 1, wherein the plurality of capillaries are substantially parallel.

3. The microfluidic passive sample separation device according to claim 1, wherein each capillary has a width of about 8 μm to about 20 μm and is spaced about 8 μm to about 80 μm from an adjacent capillary; or each capillary has a width of about 12 μm and is spaced about 40 μm from an adjacent capillary.

4. The microfluidic passive sample separation device according to claim 1, wherein the capillary micropump has at least a first end and a second end opposite the first end, and wherein the width of the micropump increases and then decreases between the first end and the second end.

5. The microfluidic passive sample separation device according to claim 1, wherein the capillary micropump comprises:
an elliptical, half-hexagonal, or hexagonal capillary micropump; and/or
a staggered array of substantially circular-, square-, rectangular-, or oval-shaped microstructures.

6. The microfluidic passive sample separation device according to claim 1, wherein the capillary micropump comprises:
a hexagonal capillary micropump; and/or
a staggered array of substantially oval-shaped microstructures.

7. The microfluidic passive sample separation device according to claim 1, wherein the filter is a filter membrane.

8. The microfluidic passive sample separation device according to claim 7, wherein the filter membrane has:
a thickness of about 1 μm to about 20 μm, or about 9 μm; and/or a pore size of about 0.3 μm to about 10 μm; or about 0.6 μm.

9. The microfluidic passive sample separation device according to claim 1, wherein the microfluidic passive filtration device is inverted or non-inverted.

10. The microfluidic passive sample separation device according to claim 1, wherein the inlet defines a volume of at least 50 μL, or at least 80 μL, or between about 50 μL to about 200 μL.

11. The microfluidic passive sample separation device according to claim 1, wherein the liquid sample comprises blood; whole blood; urine; seminal fluid; vaginal fluid; tissue; an aqueous solution; marine or freshwater water; a marine or freshwater plant; marine or freshwater food; phytoplankton; shellfish; food; or milk; or a combination thereof.

12. The microfluidic passive sample separation device according to claim 1, wherein:
the filtrate comprises an analyte, and
the analyte optionally comprises a nucleic acid, DNA, circulating DNA, high-risk HPV circulating DNA, high-risk HPV16 circulating DNA, a protein, an antibody, an antigen, a metabolite; a toxin; a biotoxin, such as domoic acid or okadaic acid; a small molecule; a cell; a bacterium; a virus; or an inflammatory marker, such as interleukins, cytokines, creative protein, pro-calcitonin, or pre-sepsin.

13. The microfluidic passive sample separation device according to claim 1, wherein the filtrate comprises plasma.

14. The microfluidic passive sample separation device according to claim 1, wherein the microfluidic passive plasma separation device is suitable for point-of-care use and/or sized to be portable by a single user.

15. The microfluidic passive sample separation device according to claim 1, further comprising a capillary microchannel and a capillary microwell housed in the body,
the microchannel having a first end fluidly connected to the inlet for receiving the liquid sample, and a second end fluidly connected to the microwell for dispensing the liquid sample to the microwell, the microchannel configured to move the liquid sample from the inlet to the microwell by capillary force, and
the microwell having a first end fluidly connected to the microchannel for receiving the liquid sample, and a second end coupled to the filter, the microwell configured to move the liquid sample from the microchannel to the filter by capillary force.

16. The microfluidic passive sample separation device of claim 1, further comprising a sensor for detecting an analyte, the sensor being fluidly connected to the outlet, and the sensor optionally comprising an electrochemical sensor, an optical sensor, a piezoelectric sensor, or a photodetector.

17. A detection device for detecting an analyte in a liquid sample, the detection device comprising:
a microfluidic passive sample separation device comprising:
a body defining an inlet and an outlet, the body housing
a filter for filtering a liquid sample in contact therewith and for producing a filtrate;
a plurality of capillaries configured to withdraw the filtrate from the filter by capillary force, each capillary having
a first end fluidly connected to the filter for receiving the filtrate; and
a second end fluidly connected to a capillary micropump for dispensing the filtrate to the capillary micropump;
the capillary micropump being configured to receive the filtrate and to pump the filtrate to the outlet; and
an electrochemical biosensor, the electrochemical biosensor fluidly coupled to the microfluidic passive sample separation device for detecting the analyte in the filtrate.

18. The detection device according to claim 17, wherein the electrochemical biosensor comprises a working electrode having a surface comprising a probe configured to couple to, bind to, or hybridize with the analyte.

19. The detection device according to claim 18, wherein the working electrode is a carbon-based electrode, gold-based electrode, a graphene-based electrode, or a screen-printed electrode.

20. The detection device according to any one of claims 18, wherein the probe is immobilized on the working electrode by drop-cast graphene oxide (GO) on the surface of the working electrode and a covalent bond formed between the probe and the GO; and the probe is optionally a complementary single stranded DNA (cssDNA).

21. The detection device according to claim 17, wherein the analyte comprises a nucleic acid, DNA, circulating DNA, high-risk HPV circulating DNA, high-risk HPV16 circulating DNA, a protein, an antibody, an antigen, a metabolite; a toxin; a biotoxin, such as domoic acid or okadaic acid; a small molecule; a cell; a bacterium; a virus; or an inflammatory marker, such as interleukins, cytokines, creative protein, pro-calcitonin, or pre-sepsin.

22. The detection device according to claim 17, wherein the liquid sample comprises blood; whole blood; urine; seminal fluid; vaginal fluid; tissue; an aqueous solution; marine or freshwater water; a marine or freshwater plant; marine or freshwater food; phytoplankton; shellfish; food; or milk; or a combination thereof.

23. The detection device according to claim 17, wherein the filtrate comprises plasma.

24. The detection device according to claim 17, wherein the microfluidic passive sample separation device is inverted.

25. The detection device according to claim 17, wherein the microfluidic passive sample separation device further comprises a capillary microchannel and a capillary microwell housed in the body,
the microchannel having a first end fluidly connected to the inlet for receiving the liquid sample, and a second end fluidly connected to the microwell for dispensing the liquid sample to the microwell, the microchannel configured to move the liquid sample from the inlet to the microwell by capillary force, and
the microwell having a first end fluidly connected to the microchannel for receiving the liquid sample, and a second end coupled to the filter, the microwell configured to move the liquid sample from the microchannel to the filter by capillary force.

26. The detection device according to claim 17, wherein the detection device is suitable for point-of-care use and/or sized to be portable by a single user.

27. A microfluidic passive sample separation device comprising
a body defining an inlet and an outlet, the inlet defining a volume and configured to accept a liquid sample,
the body housing:
a capillary microchannel, a capillary microwell, and a filter,
the microchannel having a first end fluidly connected to the inlet for receiving the liquid sample, and a second end fluidly connected to the microwell for dispensing the liquid sample to the microwell, the microchannel configured to move the liquid sample from the inlet to the microwell by capillary force,
the microwell having a first end fluidly connected to the microchannel for receiving the liquid sample, and a second end coupled to a filter, the microwell configured to move the liquid sample from the microchannel to the filter by capillary force,
the filter configured to filter the liquid sample received from the microwell to produce a filtrate; and
a plurality of capillaries and a capillary micropump,
the plurality of capillaries configured to withdraw the filtrate from the filter by capillary force, each capillary having a first end fluidly connected to the filter for receiving the filtrate;
and a second end fluidly connected to the capillary micropump for dispensing the filtrate to the capillary micropump;
the capillary micropump being configured to receive the filtrate and to pump the filtrate through to the outlet.

28. The device according to claim 27, wherein the filter is:
vertically displaced relative to the microchannel; and/or.
parallel to the microchannel.

29. The device according to claim 27, wherein the outlet is:
vertically displaced relative to the inlet; and/or
parallel to the inlet.

30. The device according to claim 27, wherein the outlet comprises a series of outlet reservoirs.

31. The device according to claim 27, wherein
the capillary microchannel has a width between about 100 μm to about 1 mm; and/or
the microwell has a width between about 1 mm to about 5 mm.

32. The device according to claim 27, wherein the plurality of capillaries are substantially parallel; and wherein optionally each capillary has a width of about 8 μm to about 20 μm and is spaced about 8 μm to about 80 μm from an adjacent capillary; or each capillary has a width of about 12 μm and is spaced about 40 μm from an adjacent capillary.

33. The device according to claim 27, wherein the capillary micropump has at least a first end and a second end opposite the first end, and wherein the width of the micropump increases and then decreases between the first end and the second end.

34. The device according to claim 27, wherein the capillary micropump comprises:
an elliptical, half-hexagonal, or hexagonal capillary micropump; and/or
a staggered array of substantially circular-, square-, rectangular-, or oval-shaped microstructures.

35. The device according to claim 27, wherein the microfluidic passive filtration device is non-inverted.

36. The device according to claim 27, wherein the liquid sample comprises blood; whole blood; urine; seminal fluid; vaginal fluid; tissue; an aqueous solution; marine or freshwater water; a marine or freshwater plant; marine or freshwater food; phytoplankton; shellfish;
food; or milk; or a combination thereof.

37. The device according to claim 27, wherein:
the filtrate comprises an analyte, and
the analyte optionally comprises a nucleic acid, DNA, circulating DNA, high-risk HPV circulating DNA, high-risk HPV16 circulating DNA, a protein, an antibody, an antigen, a metabolite; a toxin; a biotoxin, such as domoic acid or okadaic acid; a small molecule; a cell; a bacterium; a virus; or an inflammatory marker, such as interleukins, cytokines, creative protein, pro-calcitonin, or pre-sepsin.

38. The device according to claim 27, further comprising a sensor for detecting an analyte, the sensor being fluidly connected to the outlet, and the sensor optionally comprising an electrochemical sensor, an optical sensor, a piezoelectric sensor, or a photodetector.


SEQUENCE LISTING (3 sequences)

SEQ ID NO: 1 - DNA, 26 bp, synthetic construct
  source 1..26: mol_type = other DNA
  misc_feature 26: note = tagged with amino modifier (AmMO)
gggctctgtccggttctgcttgtcca

SEQ ID NO: 2 - DNA, 26 bp, synthetic construct
  source 1..26: mol_type = other DNA
tggacaagcagaaccggacagagccc

SEQ ID NO: 3 - DNA, 30 bp, synthetic construct
  source 1..30: mol_type = other DNA
  misc_feature 1: note = tagged with cyanine 3 (Cy3)
ggtggtggggggggttggtagggtgtcttc